(19) United States
(12) Patent Application Publication
(10) Pub. No.: US 2023/0028989 A1
(43) Pub. Date: Jan. 26, 2023

(54) CELL INJECTION PRINTING WITH A COAXIAL NEEDLE

(71) Applicant: Scripps Health, San Diego, CA (US)

(72) Inventors: Darryl D. D'LIMA, San Diego, CA (US); Clifford W. COLWELL, La Jolla, CA (US)

(21) Appl. No.: 17/782,010

(22) PCT Filed: Dec. 2, 2020
(86) PCT No.: PCT/US2020/062805

§ 371 Date: Jun. 2, 2022

Related U.S. Application Data
(60) Provisional application No. 62/943,081, filed on Dec. 3, 2019.

(51) Int. Cl. A61M 5/32; C12N 5/077

(52) U.S. Cl. CPC A61M 5/329; C12N 5/0655; A61M 2205/0266

(57) ABSTRACT

Provided are an apparatus and method for injection of a fluid into a substrate. In some embodiments, an apparatus or method described herein delivers cells to a tissue scaffold, graft, or site of tissue injury to facilitate the repair of a tissue defect.

### CROSS-REFERENCE TO RELATED APPLICATIONS

This application claims the benefit of U.S. Provisional Application No. 62/943,081, filed Dec. 3, 2019, the entirety of which is incorporated herein by reference.

### BACKGROUND

Cartilage is a type of flexible connective tissue commonly found in articulating surfaces of joints. Tears can occur when excessive forces are applied to cartilage. Cartilaginous tissues display minimal healing due to limited vascularization and an inability of cells to infiltrate through cartilage. Current treatment strategies which aim to recellularize cartilage through the direct injection of cells are often ineffective due to the shortcomings of traditional injection methods.

Many traditional injection methods discharge fluid from a single, static opening located at the distal end of a needle. Discharge through a single, static opening can cause the flow of fluid to be dependent almost entirely on the modulation of the fluid flow rate through the needle. When fluid is injected to dense, compressive substrates using traditional methods, the compressive forces of the substrate can force fluid out of the injection site and render the delivery of fluid to the substrate ineffective. Even if fluid injected via traditional methods does remain in a substrate, such fluid tends to be confined to a small area such that the fluid delivery is ineffective for the healing of a larger wound. Thus, improved delivery strategies are needed to recellularize cartilaginous tissues in the area of a tear in order to promote the healing process.

### SUMMARY

Provided herein are devices and methods for delivering cells to a dense substrate. In one aspect, the present disclosure provides coaxial needles comprising: a proximal end, a distal end, and a first elongate tubular body and a second elongate tubular body that are arranged concentrically, wherein the first elongate tubular body and the second elongate tubular body comprise: a cylindrical side wall forming a lumen that extends along a longitudinal axis between the proximal end and the distal end, and a plurality of side wall openings arranged along the cylindrical side wall; wherein the second elongate tubular body is movable relative to the first elongate tubular body to form a plurality of configurations, wherein at least one of the plurality of configurations comprises a paired opening formed by an overlap of at least one of the plurality of side wall openings of the first elongate tubular body and at least one of the plurality of side wall openings of the second elongate tubular body.

In some embodiments, the paired opening is formed by one of the plurality of side wall openings of the first elongate tubular body and one of the plurality of sidewall openings of the second elongate tubular body. In some embodiments, the second elongate tubular body is arranged concentrically within the lumen of the first elongate tubular body. In some embodiments, the first elongate tubular body is arranged concentrically within the lumen of the second elongate tubular body.

In some embodiments, the lumen of the first elongate tubular body has a diameter of 0.5 mm to 500 mm. In some embodiments, the lumen of the first elongate tubular body has a diameter of 0.1 mm to 400 mm. In some embodiments, the lumen of the second elongate tubular body has a diameter of about 0.1 mm to about 400 mm. In some embodiments, the lumen of the second elongate tubular body has a diameter of 0.5 mm to 500 mm. In some embodiments, the first elongate tubular body has a length of 0.5 cm to 200 cm. In some embodiments, the second elongate tubular body has a length of 0.5 cm to 200 cm.

In some embodiments, the coaxial needle further comprises a piercing member located on the distal end and attached to the second elongate tubular body. In some embodiments, the coaxial needle further comprises a piercing member located on the distal end and attached to the first elongate tubular body. In some embodiments, the coaxial needle further comprises an opening located on the distal end.

In some embodiments, the plurality of side wall openings of the first elongate tubular body is arranged in a random, linear, spiral, radial, Fibonacci spiral, logarithmic spiral, Archimedean spiral, zig-zag, or checkerboard pattern. In some embodiments, the plurality of openings of the first elongate tubular body is arranged in a pattern, wherein the pattern replicates the spacing of cells in a biological tissue. In some embodiments, the plurality of side wall openings of the second elongate tubular body is arranged in a random, linear, spiral, radial, Fibonacci spiral, logarithmic spiral, Archimedean spiral, zig-zag, or checkerboard pattern. In some embodiments, the plurality of openings of the second elongate tubular body is arranged in a pattern, wherein the pattern replicates the spacing of cells in a biological tissue.

In some embodiments, an area of the paired opening is at most 80 mm². In some embodiments, at least one of the plurality of side wall openings of the first elongate tubular body or at least one of the plurality of side wall openings of the second elongate tubular body has an area of 0.1 mm² to 80 mm².

In some embodiments, at least one of the plurality of side wall openings of the first elongate tubular body has an elliptical shape, a circular shape, a rectangular shape, a triangular shape, or a tear drop shape. In some embodiments, at least one of the plurality of side wall openings of the second elongate tubular body has an elliptical shape, a circular shape, a rectangular shape, a triangular shape, or a tear drop shape.

In some embodiments, the coaxial needle further comprises an expanding member located on or adjacent to the distal end of the coaxial needle, wherein the expanding member is a metallic material and the metallic material comprises a shape memory alloy. In some embodiments, the cylindrical side wall of the first elongate tubular body and the cylindrical side wall of the second elongate tubular body are a metallic material, wherein the metallic material comprises a shape memory alloy. In some embodiments, the shape memory alloy is nitinol.

In some embodiments, the second elongate tubular body is movable in the axial direction. In some embodiments, the second elongate tubular body can rotate about the longitudinal axis of the second elongate tubular body.

Also provided herein are methods of injecting cells into a substrate comprising: inserting a coaxial needle into the substrate, wherein the coaxial needle comprises: a proximal end, a distal end, and an inner elongate tubular body and an outer elongate tubular body that are arranged concentrically, wherein the inner elongate tubular body and the outer elongate tubular body comprise: a cylindrical side wall forming a lumen that extends along a longitudinal axis between the proximal end and the distal end, and a plurality of side wall openings arranged along the cylindrical side wall; wherein at least one of the inner or outer elongate tubular bodies is movable relative to the other to form a plurality of configurations, wherein at least one of the plurality of configurations comprises a paired opening formed by an overlap of at least one of the plurality of side wall openings of the inner elongate tubular body and at least one of the plurality of side wall openings of the outer elongate tubular body; and flowing a fluid comprising a plurality of cells into the lumen of the inner elongate tubular body such that the fluid passes through the paired opening and into the substrate.

In some embodiments, the paired opening is formed by one of the plurality of side wall openings of the first elongate tubular body and one of the plurality of sidewall openings of the second elongate tubular body.

In some embodiments, the inner elongate tubular body is movable relative to the outer elongate tubular body. In some embodiments, the inner elongate tubular body moves in the axial direction in relation to the outer elongate tubular body. In some embodiments, the inner elongate tubular body rotates about the longitudinal axis of the inner elongate tubular body.

In some embodiments, the outer elongate tubular body is movable relative to the inner elongate tubular body. In some embodiments, the outer elongate tubular body moves in the axial direction in relation to the inner elongate tubular body. In some embodiments, the outer elongate tubular body rotates about the longitudinal axis of the outer elongate tubular body.

In some embodiments, the method further comprises controlling a position of the outer elongate tubular body relative to the inner elongate tubular body. In some embodiments, the method further comprises controlling a position of the inner elongate tubular body relative to the outer elongate tubular body.

In some embodiments, the plurality of openings of the inner elongate tubular body is arranged in a pattern, wherein the pattern replicates the spacing of cells in a biological tissue. In some embodiments, the plurality of side wall openings of the inner elongate tubular body is arranged in random, linear, spiral, radial, Fibonacci spiral, logarithmic spiral, Archimedean spiral, zig-zag, or checkerboard pattern. In some embodiments, the plurality of side wall openings of the outer elongate tubular body is arranged in a random, linear, spiral, radial, Fibonacci spiral, logarithmic spiral, Archimedean spiral, zig-zag, or checkerboard pattern. In some embodiments, the plurality of openings of the outer elongate tubular body is arranged in a pattern, wherein the pattern replicates the spacing of cells in a biological tissue.

In some embodiments, an area of the paired opening is at most 80 mm².

In some embodiments, the fluid comprises a plurality of cells. In some embodiments, the plurality of cells comprises chondrogenic cells, chondrogenic precursors, multipotent cells, or pluripotent cells. In some embodiments, the fluid flows through an opening located on the distal end of the coaxial needle. In some embodiments, the opening located on the distal end of the coaxial needle increases in size after being inserted into the substrate. In some embodiments, the coaxial needle changes shape after being inserted into the substrate.

In some embodiments, the substrate is a biological tissue. In some embodiments, the biological tissue is human tissue, orthopedic tissue, cartilage, or devitalized tissue. In some embodiments, the substrate is an acellular scaffold.

Also provided herein are methods of repairing damaged tissue comprising: implanting an acellular tissue scaffold into the damaged tissue, inserting a coaxial needle into the acellular tissue scaffold, wherein the coaxial needle comprises: a proximal end, a distal end, and an inner elongate tubular body and an outer elongate tubular body that are arranged concentrically, wherein the inner elongate tubular body and the outer elongate tubular body comprise: a cylindrical side wall forming a lumen that extends along a longitudinal axis between the proximal end and the distal end, and a plurality of side wall openings arranged along the cylindrical side wall; wherein at least one of the inner or outer elongate tubular bodies is movable relative to the other to form a plurality of configurations, wherein at least one of the plurality of configurations comprises a paired opening formed by an overlap of at least one of the plurality of side wall openings of the inner elongate tubular body and at least one of the plurality of side wall openings of the outer elongate tubular body; and flowing a fluid comprising a plurality of cells into the lumen of the inner elongate tubular body such that the fluid passes through the paired opening and into the acellular tissue scaffold.

In some embodiments, the paired opening is formed by one of the plurality of side wall openings of the first elongate tubular body and one of the plurality of sidewall openings of the second elongate tubular body.

In some embodiments, the inner elongate tubular body is movable relative to the outer elongate tubular body. In some embodiments, the inner elongate tubular body moves in the axial direction in relation to the outer elongate tubular body. In some embodiments, the inner elongate tubular body rotates about the longitudinal axis of the inner elongate tubular body.

In some embodiments, the outer elongate tubular body is movable relative to the inner elongate tubular body. In some embodiments, the outer elongate tubular body moves in the axial direction in relation to the inner elongate tubular body. In some embodiments, the outer elongate tubular body rotates about the longitudinal axis of the outer elongate tubular body.

In some embodiments, the method further comprises controlling a position of the outer elongate tubular body relative to the inner elongate tubular body. In some embodiments, the method further comprises controlling a position of the inner elongate tubular body relative to the outer elongate tubular body.

In some embodiments, the plurality of side wall openings of the inner elongate tubular body is arranged in a random, linear, spiral, radial, Fibonacci spiral, logarithmic spiral, Archimedean spiral, zig-zag, or checkerboard pattern. In some embodiments, the plurality of openings of the inner elongate tubular body is arranged in a pattern, wherein the pattern replicates the spacing of cells in a biological tissue. In some embodiments, the plurality of side wall openings of the outer elongate tubular body is arranged in a random, linear, spiral, radial, Fibonacci spiral, logarithmic spiral, Archimedean spiral, zig-zag, or checkerboard pattern. In some embodiments, the plurality of openings of the outer elongate tubular body is arranged in a pattern, wherein the pattern replicates the spacing of cells in a biological tissue.

In some embodiments, an area of the paired opening is at most 80 mm².

In some embodiments, the fluid comprises a plurality of cells. In some embodiments, the plurality of cells comprises chondrogenic cells, pluripotent cells, multipotent cells, or chondrogenic precursors. In some embodiments, the fluid flows through an opening located on the distal end of the coaxial needle. In some embodiments, the opening located on the distal end of the coaxial needle increases in size after being inserted into the substrate. In some embodiments, the coaxial needle changes shape after being inserted into the substrate.

In some embodiments, the acellular tissue scaffold is a devitalized tissue allograft.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows an embodiment of a coaxial needle of the disclosure.

FIG. 2 shows a depiction of a shape memory alloy coaxial needle of the disclosure that changes shape with a change in temperature.

FIG. 3 illustrates an example of coaxial needle of the disclosure being used to deliver cells to torn tissue.

FIG. 4 shows a schematic of the process of filling a tissue defect with an acellular substrate and then cellularizing the substrate with a coaxial needle of the disclosure.

FIG. 5 illustrates several configurations of a coaxial needle of the disclosure.

FIG. 6 shows a depiction of a coaxial needle of the disclosure with an expanding member on the distal end that changes shape upon a temperature change.

### DETAILED DESCRIPTION

Provided herein are devices and methods useful in promoting tissue healing. In some embodiments, devices and methods of the disclosure facilitate the wound healing process by promoting the production of extracellular matrix at the wound site.

Disclosed herein are delivery devices and methods of using the same to facilitate the delivery of fluid containing material such as cells into a substrate. Through practice of the disclosure, one can effectively deliver cells into a dense substrate in a manner that allows for a precise distribution of cells throughout the substrate. In some embodiments, the devices and methods deliver materials such as cells to, for example, wounded tissue or acellular tissue scaffolds that can be implanted into a wound site.

Cellularization of damaged tissue plays an important role in the healing process. During the wound healing process, cells accumulate at the wound site and produce extracellular matrix (ECM). ECM production facilitates wound closing and repair. Cartilaginous tissue, however, displays poor healing properties. Tissue injury can damage or kill chondrocytes, the predominant cell type in cartilage. Cartilage has limited vascularity combined with high density, which limits the ability of new chondrocytes to reach the site of injury. This leads to an acellular wound site and ultimately leaves frayed and fragmented tissue that does not get repaired. Thus, cellularization of a wound site allows for the production of ECM across the wound, facilitating wound closing and repair.

One method by which cells are delivered to tissues is through the direct injection of cells. Some strategies of using cells to facilitate the wound healing process evenly distribute the cells around the site of injury. Unfortunately, it can be difficult to obtain an even distribution of cells around the site of injury in a dense tissue such as cartilage because the density of the tissue can render the cells mostly immobile following injection. Furthermore, compressive forces in tissues (e.g. cartilage) can force injected cells out of the tissue altogether such that the injected cells cannot be stabilized in the tissue.

An alternative method to cellularize damaged tissue is through the implantation of cellularized scaffolds or cellularized tissue grafts. Once implanted, tissue grafts or scaffolds can facilitate wound healing by integrating with the surrounding tissue. Integration of tissue grafts and scaffolds with native tissue can be facilitated by the production of ECM by cells contained within the scaffolds or grafts. A homogenous distribution of ECM producing cells throughout a scaffold or tissue graft is often beneficial to tissue integration; as such a distribution encourages integration of the entire surface area of the scaffold or graft. However, tissue grafts and scaffolds are frequently designed to possess mechanical properties that match those of the intended implantation site. Thus, many of the cell delivery difficulties that exist in dense, compressive tissue such as cartilage also exist in the tissue grafts and scaffolds intended for implantation into such tissues.

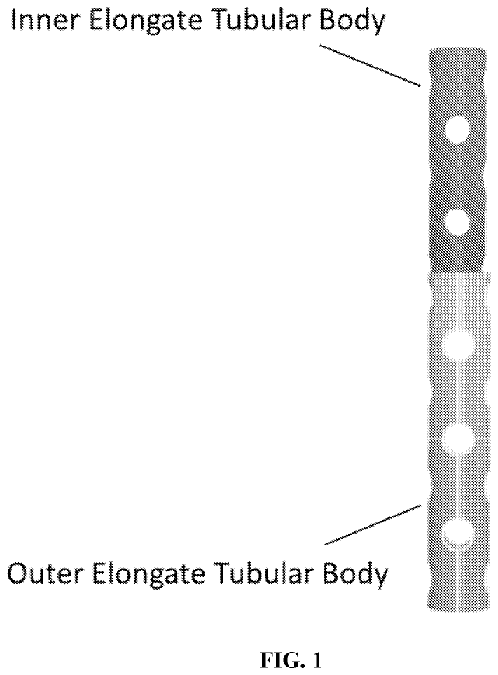

Use of methods or devices of the disclosure allows for the discharge of fluid (and materials contained in the fluid, such as cells) from a needle in a controlled manner and addresses the previously mentioned shortcomings of traditional injection methods. The methods and devices can achieve controlled discharge of cells via the utilization of a coaxial needle with one or more side wall openings, which can be located along the length of the concentrically arranged inner and outer elongate tubular bodies, as shown in FIG. 1. The tubular bodies of the coaxial needles can rotate about their longitudinal axis. Alternatively or in addition, the tubular bodies of the coaxial needles can also be configured to move in the axial direction. Through relative movement of the elongate tubular bodies, the arrangement of the side wall openings can be controllable. Paired openings result from an overlap of side wall openings on the inner and outer elongate tubular bodies and provide fluidic communication from the inner lumen of the coaxial needle to the outside environment. The paired openings of a coaxial needle can be used to simultaneously discharge fluid at multiple locations along the length of the needle. Further, utilization of paired openings allows for control of the discharge of fluid in a specific direction or at a specific location or locations. The flow of fluid out of a coaxial needle of the disclosure can be controllable through modulation of the flow rate of fluid into the needle, the size of paired openings via controlled movement of the elongate tubular bodies, and the positions of the paired openings relative to the injection site.

By utilizing a coaxial needle described herein, devices and methods of the disclosure are able to inject cells homogenously and in a controlled manner along the length of a tissue injury or throughout a tissue scaffold or graft. Ejection of cells out of paired-openings on the side of a needle allows for the cells to integrate into the substrate prior to the removal of the needle and the exertion of compressive forces on the fluid. Delivery of cells in this manner allows the cells to integrate homogenously around a tissue injury site or throughout a scaffold or graft and aid in the wound healing process.

### I. Coaxial Needles

In some embodiments, coaxial needles described herein comprise two or more elongate tubular bodies arranged concentrically. An elongate tubular body can comprise a cylindrical side wall extending from a proximal end to a distal end and forming a lumen. In some embodiments, an elongate tubular body is arranged concentrically within a second elongate tubular body to form inner and outer elongate tubular bodies. Each of the inner and outer elongate tubular bodies can comprise a plurality of side wall openings arranged along its cylindrical side wall. Inner and outer elongate bodies with side wall openings can be configured to be moveable relative to each other. In some configurations, a side wall opening on the inner elongate tubular body and a side wall opening on the outer elongate tubular body overlap to form a paired opening. Paired openings can provide a flow path from the lumen of an inner elongate tubular body to the outside environment. By controlling the relative movement of inner and/or outer elongate tubular bodies, a user of the coaxial needle can control the flow of fluid out of the coaxial needle via control of the number, size, and/or location of paired openings.

In some embodiments, a coaxial needle of the disclosure comprises a piercing member located on the distal end and attached to at least one of the inner elongate tubular body and the outer elongate tubular body. In some embodiments, a coaxial needle of the disclosure comprises an opening located on the distal end of the coaxial needle.

In some embodiments, the coaxial needle comprises an expanding member. Expanding members can increase or decrease in size depending on the condition (e.g., temperature, rigidity, pressure, etc.) of the surrounding environment.

In some embodiments, a coaxial needle and/or the component parts of a coaxial needle are disinfected or sterile. In some instances, a coaxial needle and/or the component parts of a coaxial needle are sterilized or disinfected by, for example, cleaning with a chemical agent, cleaning with soap, placement in an autoclave, heat sterilization, sterilization with ultraviolet light, or a combination of any of the foregoing.

A. Tubular Bodies

The coaxial needles can comprise a first elongate tubular body that is moveable relative to a second elongate tubular body. In some embodiments, a coaxial needle of the disclosure has: i) an inner elongate tubular body that is moveable and an outer elongate tubular body that is stationary; ii) an outer elongate tubular body that is moveable and the inner elongate tubular body is stationary; or iii) outer and inner elongate tubular bodies that are each independently moveable. Movement of an elongate tubular body occurs in, for example, an axial direction (i.e. lengthwise between the proximal and distal end of the tubular bodies), or a radial (i.e. the body rotates) direction. In some instances, movement of an elongate tubular body of a coaxial needle disclosed herein is controllable by, for example, a user or computer program.

The arrangement of an inner elongate tubular body relative to an outer elongate tubular body can be determined by the structure of the coaxial needle. In some embodiments, the inner elongate tubular body and the outer elongate tubular body are each connected independently at the proximal end of the tubular body to a coupler. Inner and outer elongate tubular bodies can each be connected to an independent coupler or both an inner and outer elongate tubular body can be connected to a common coupler. A coupler can, for example, connect tubular bodies to a fluid injection system. Elongate tubular bodies can fit into the first end of a coupler while a second end of a coupler fits into an end of a fluid injection system. Non-limiting examples of couplers include needle hubs, Leur lock fittings (male or female), flanges, quick-connect fittings, and threaded components. In some embodiments, an elongate tubular body comprises threading or a Leur lock fitting (male or female) to facilitate connection to a coupler. In some embodiments, the position of elongate tubular bodies can be controlled via the coupler. For example, user or computer-controlled movement of a coupler can cause an inner or outer elongate tubular body to move in an axial or radial direction. In some embodiments, the inner elongate tubular body and outer elongate tubular body have a sliding fit. For example, the inner elongate tubular body can remain within the outer elongate tubular body due to friction between the cylindrical side walls of the tubular bodies. In some embodiments, tubular bodies connected with a sliding fit can move relative to the other tubular body. In some embodiments, an inner elongate tubular body and an outer elongate tubular body are held in place via a bearing (e.g. a rotary bearing, a ball bearing, a roller bearing, or a plain bearing).

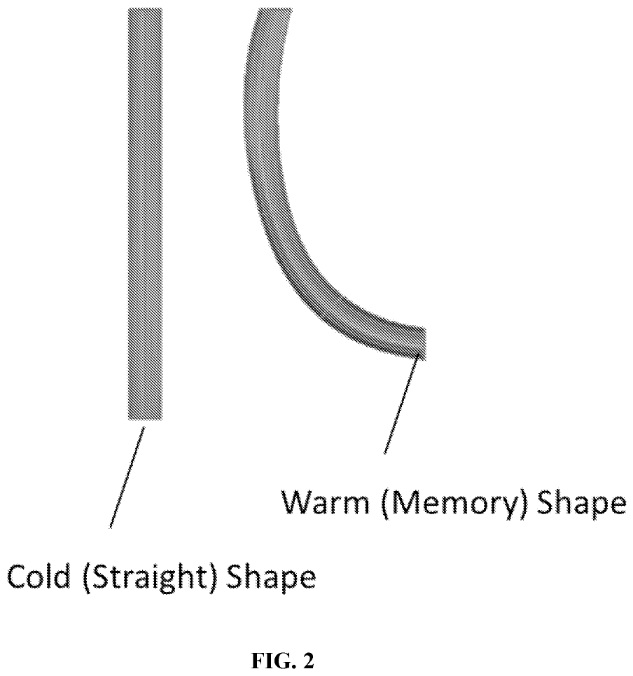

In some embodiments, the coaxial needle has an inner elongate tubular body, an outer elongate tubular body, or both an inner and outer elongate tubular body made up of a metallic material comprising a shape memory alloy such as nitinol (a metal alloy of nickel and titanium). Shape memory alloys such as nitinol can be deformed at or below a transformation temperature and can recover their original or pre-deformed shape when heated above the transformation temperature. Coaxial needles with tubular bodies composed of, or comprising, shape memory alloys (i.e. shape memory alloy coaxial needles) can facilitate the delivery of fluid to areas that are not easily accessible to straight needles. For example, a shape memory alloy coaxial needle at an initial temperature can be injected into a substrate in a straightened orientation. Insertion of the needle into the substrate then causes a change in the temperature of the needle causing it to bend into a new shape as shown in FIG. 2. The change in shape of the needle can allow delivery of fluid to an area of the substrate that is different than the area that fluid would have been delivered to had the needle retained its original shape.

Shape memory alloy coaxial needles can be designed so that the needle curves at different angles after insertion into a substrate (e.g., 180 degrees, 150 degrees, 130 degrees, 120 degrees, 90 degrees, 60 degrees, 30 degrees, or 10 degrees) relative to the orientation of the outer or inner tubular body prior to insertion into the substrate, or at different directions (e.g., left, right, etc.) once inserted into the substrate. In some embodiments, a shape memory alloy coaxial needle is designed to curve between 0 degrees to 180 degrees, 0 degrees to 150 degrees, 0 degrees to 120 degrees, 0 degrees to 90 degrees, 0 degrees to 60 degrees, 0 degrees to 10 degrees, 10 degrees to 180 degrees, 10 degrees to 150 degrees, 10 degrees to 120 degrees, 10 degrees to 90 degrees, 10 degrees to 60 degrees, 10 degrees to 30 degrees, 30 degrees to 180 degrees, 30 degrees to 150 degrees, 30 degrees to 120 degrees, 30 degrees to 90 degrees, 30 degrees to 60 degrees, 60 degrees to 180 degrees, 60 degrees to 150 degrees, 60 degrees to 120 degrees, 60 degrees to 90 degrees, 90 degrees to 180 degrees, 90 degrees to 150 degrees, 90 degrees to 120 degrees, 120 degrees to 180 degrees, 120 degrees to 150 degrees, or 150 degrees to 180 degrees. In some embodiments, shape memory alloy coaxial needles change shape in response to a temperature increase. In some embodiments, shape memory alloy coaxial needles change shape in response to a temperature decrease.

In some embodiments, a coaxial needle comprises an inner elongate tubular body comprising a shape memory alloy which can drive movement of a flexible outer elongate tubular body. In some embodiments, a coaxial needle comprises an outer elongate tubular body comprising a shape memory alloy which can drive movement of a flexible inner elongate tubular body. In some embodiments, a coaxial needle comprises an inner elongate tubular body and an outer elongate tubular body that are both comprised of a shape memory alloy such as nitinol.

The temperature dependent properties of nitinol can be affected by the nickel-titanium ratio. In some embodiments, the coaxial needles comprise nitinol with a nickel:titanium ratio of between 0.8:1 and 1.2:1. In some embodiments, the nickel:titanium ratio of nitinol is about 0.8, about 0.81, about 0.82, about 0.83, about 0.84, about 0.85, about 0.86, about 0.87, about 0.88, about 0.89, about 0.9, about 0.91, about 0.92, about 0.93, about 0.94, about 0.95, about 0.96, about 0.97, about 0.98, about 0.99, about 1, about 1.01, about 1.02, about 1.03, about 1.04, about 1.05, about 1.06, about 1.07, about 1.08, about 1.09, about 1.1, about 1.11, about 1.12, about 1.13, about 1.14, about 1.15, about 1.16, about 1.17, about 1.18, about 1.19, or about 1.2.

B. Side Wall Openings

In some embodiments, an elongate tubular body of the disclosure comprises a plurality of side wall openings arranged along the cylindrical side wall of the elongate tubular body. Side wall openings can be arranged along the entire length of a cylindrical side wall or along a portion of the side wall. For example, an elongate tubular body can have side wall openings along the distal half of the body but not along the proximal half of the body; along the proximal half of the body but not the distal half; or according to a pattern, such as alternating regions with and without side wall openings.

Several characteristics of side wall openings affect the delivery of fluid from a coaxial needle. The characteristics include, for example, the number, shape, size, and arrangement of side wall openings.

In some embodiments, all of the side wall openings arranged along a cylindrical side wall are of the same shape, while in further embodiments a cylindrical side wall has side wall openings of multiple shapes. Non-limiting examples of shapes of side wall openings include elliptical, circular, triangular, rectangular, and tear drop shaped.

Side wall openings can be arranged in different densities and arrangements along cylindrical side walls. The arrangement and/or density of side wall openings along a cylindrical side wall of an elongate tubular body can affect the discharge of fluid from a coaxial needle disclosed herein. In some embodiments, side wall openings of the disclosure are randomly arranged along a cylindrical side wall, while in other embodiments, side wall openings are arranged in a pattern. For example, side wall openings of the disclosure are, in some embodiments, arranged in a linear pattern, a radial pattern, a zig-zag patter, a checkerboard pattern, a spiral pattern, or a mathematic sequence pattern such as a Fibonacci spiral, an Archimedean spiral, a logarithmic spiral, or in a combination of any of the foregoing patterns.

In some embodiments, the pattern in which side wall openings are arranged changes throughout the length of the cylindrical side wall. For example, the side wall openings of the disclosure are, in some embodiments, arranged to replicate the arrangement of cells in a biological tissue such as cartilage. In cartilage tissue, cells in the deep zone, (located just above the junction between cartilage and bone) are arranged in columns, touching or almost touching each other; cells in the middle zone (located above the deep zone) are arranged randomly or somewhat randomly; and cells in the superficial zone are arranged roughly perpendicular to cell columns in the deep zone. In further embodiments, side wall openings of the disclosure are arranged to mimic the arrangement of cells in muscle, ligament, tendon, bone, skin, hepatic, renal, splenic, lung, cardiac, stomach, or intestinal tissue.

In some coaxial configurations, the arrangement of side wall openings on the inner elongate tubular body can be different or the same as the arrangement of side wall openings on the outer elongate tubular body. Such arrangements can be tailored to provide for different patterns of paired openings during operation.

In some embodiments, the number of side wall openings on an inner elongate tubular body of a coaxial needle is the same as the number of side wall openings on an outer elongate tubular body of the coaxial needle. In some embodiments, the number of side wall openings on an inner elongate tubular body of a coaxial needle is different than the number of side wall openings on an outer elongate tubular body of the coaxial needle.

In some embodiments, an inner elongate tubular body has 1, 2, 3, 4, 5, 6, 7, 8, 9, 10, 11, 12, 13, 14, 15, 16, 17, 18, 19, 20, 21, 22, 23, 24, 25, 50, 75, 100, 125, 150, 200, 250, 300, 350, 400, 450, 500, 600, 700, 800, 900, or 1,000 side wall openings. In some embodiments, an inner elongate tubular body has at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 50, at least 75, at least 100, at least 125, at least 150, at least 200, at least 250, at least 300, at least 350, at least 400, at least 450, at least 500, at least 600, at least 700, at least 800, at least 900, or at least 1,000 side wall openings. In some embodiments, an inner elongate tubular body has at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 21, at most 22, at most 23, at most 24, at most 25, at most 50, at most 75, at most 100, at most 125, at most 150, at most 200, at most 250, at most 300, at most 350, at most 400, at most 450, at most 500, at most 600, at most 700, at most 800, at most 900, or at most 1,000 side wall openings. In some embodiments, an inner elongate tubular body has between 1-1,000, 1-500, 1-250, 1-100, 1-50, 1-25, 25-1,000, 25-500, 25-250, 25-100, 25-50, 50-1,000, 50-500, 50-250, 50-100, 100-1,000, 100-500, 100-250, 250-1,000, 250-500, or 500-1,000 side wall openings.

In some embodiments, an outer elongate tubular body has 1, 2, 3, 4, 5, 6, 7, 8, 9, 10, 11, 12, 13, 14, 15, 16, 17, 18, 19, 20, 21, 22, 23, 24, 25, 50, 75, 100, 125, 150, 200, 250, 300, 350, 400, 450, 500, 600, 700, 800, 900, or 1,000 side wall openings. In some embodiments, an outer elongate tubular body has at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 50, at least 75, at least 100, at least 125, at least 150, at least 200, at least 250, at least 300, at least 350, at least 400, at least 450, at least 500, at least 600, at least 700, at least 800, at least 900, or at least 1,000 side wall openings. In some embodiments, an outer elongate tubular body has at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 21, at most 22, at most 23, at most 24, at most 25, at most 50, at most 75, at most 100, at most 125, at most 150, at most 200, at most 250, at most 300, at most 350, at most 400, at most 450, at most 500, at most 600, at most 700, at most 800, at most 900, or at most 1,000 side wall openings. In some embodiments, an outer elongate tubular body has between 1-1,000, 1-500, 1-250, 1-100, 1-50, 1-25, 25-1,000, 25-500, 25-250, 25-100, 25-50, 50-1,000, 50-500, 50-250, 50-100, 100-1,000, 100-500, 100-250, 250-1,000, 250-500, or 500-1,000 side wall openings.

In some embodiments, all the side wall openings arranged along a cylindrical side wall are the same size, while in further embodiments a cylindrical side wall has openings of multiple sizes. The size of side wall openings can be chosen based on various design considerations. For example, the size of side wall openings can be chosen to be small enough to limit the flow rate of cells out of the coaxial needle, but large enough to minimize the exposure of cells to mechanical forces as they flow through the openings. In some cases, the side wall openings are large enough to allow materials of interest, including cells, to pass through while small enough to prevent other materials, like debris, from passing through. The size of side wall openings can be the same or can vary between elongate tubular bodies. For example, the side wall openings of an inner elongate tubular body can have different sizes than the side wall openings of an outer elongate tubular body.

In some embodiments, a side wall opening of the disclosure has an area of 0.1 mm² to 80 mm². In some embodiments, a side wall opening of the disclosure has an area of 0.1 mm² to 0.5 mm², 0.1 mm² to 1 mm², 0.1 mm² to 5 mm², 0.1 mm² to 10 mm², 0.1 mm² to 20 mm², 0.1 mm² to 30 mm², 0.1 mm² to 40 mm², 0.1 mm² to 50 mm², 0.1 mm² to 60 mm², 0.1 mm² to 70 mm², 0.1 mm² to 80 mm², 0.5 mm² to 1 mm², 0.5 mm² to 5 mm², 0.5 mm² to 10 mm², 0.5 mm² to 20 mm², 0.5 mm² to 30 mm², 0.5 mm² to 40 mm², 0.5 mm² to 50 mm², 0.5 mm² to 60 mm², 0.5 mm² to 70 mm², 0.5 mm² to 80 mm², 1 mm² to 5 mm², 1 mm² to 10 mm², 1 mm² to 20 mm², 1 mm² to 30 mm², 1 mm² to 40 mm², 1 mm² to 50 mm², 1 mm² to 60 mm², 1 mm² to 70 mm², 1 mm² to 80 mm², 5 mm² to 10 mm², 5 mm² to 20 mm², 5 mm² to 30 mm², 5 mm² to 40 mm², 5 mm² to 50 mm², 5 mm² to 60 mm², 5 mm² to 70 mm², 5 mm² to 80 mm², 10 mm² to 20 mm², 10 mm² to 30 mm², 10 mm² to 40 mm², 10 mm² to 50 mm², 10 mm² to 60 mm², 10 mm² to 70 mm², 10 mm² to 80 mm², 20 mm² to 30 mm², 20 mm² to 40 mm², 20 mm² to 50 mm², 20 mm² to 60 mm², 20 mm² to 70 mm², 20 mm² to 80² mm, 30 mm² to 40² mm, 30 mm² to 50 mm², 30 mm² to 60 mm², 30 mm² to 70 mm², 30 mm² to 80 mm², 40 mm² to 50 mm², 40 mm² to 60 mm², 40 mm² to 70 mm², 40 mm² to 80 mm², 50 mm² to 60 mm², 50 mm² to 70 mm², 50 mm² to 80 mm², 60 mm² to 70 mm², 60 mm² to 80 mm², or 70 mm² to 80 mm².

In some embodiments a side wall opening of the disclosure has an area of 0.1 mm², 0.5 mm², 1 mm², 5 mm², 10 mm², 20 mm², 30 mm², 40 mm², 50 mm², 60 mm², 70 mm², or 80 mm². In some embodiments, a side wall opening of the disclosure has an area of at least 0.1 mm², at least 0.5 mm², at least 1 mm², at least 5 mm², at least 10 mm², at least 20 mm², at least 30 mm², at least 40 mm², at least 50 mm², at least 60 mm², or at least 70 mm². In some embodiments, a side wall opening of the disclosure has an area of at most 0.5 mm², at most 1 mm², at most 5 mm², at most 10 mm², at most 20 mm², at most 30 mm², at most 40 mm², at most 50 mm², at most 60 mm², at most 70 mm², or at most 80 mm².

C. Paired Openings

In some embodiments, the inner and outer elongate tubular bodies of a coaxial needle of the disclosure are moveable to form a configuration in which a side wall opening on the inner elongate tubular body and a side wall opening on the outer elongate tubular body overlap to form a paired opening. The number, size, and location of paired openings formed can control, for example, the flow of fluid and/or materials (e.g., cells) out of a coaxial needle of the disclosure. In some embodiments, the number, size, and location of paired openings is controlled via movement of the inner elongate tubular body, movement of the outer elongate tubular body, or movement of both the inner and outer elongate tubular bodies. In some embodiments, a coaxial needle disclosed herein can be configured to have 1, 2, 3, 4, 5, 6, 7, 8, 9, 10, 11, 12, 13, 14, 15, 16, 17, 18, 19, 20, 21, 22, 23, 24, 25, 50, 75, 100, 125, 150, 200, 250, 300, 350, 400, 450, 500, 600, 700, 800, 900, or 1,000 paired openings. In some embodiments, a coaxial needle disclosed herein can be configured to have at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 50, at least 75, at least 100, at least 125, at least 150, at least 200, at least 250, at least 300, at least 350, at least 400, at least 450, at least 500, at least 600, at least 700, at least 800, at least 900, or at least 1,000 paired openings. In some embodiments, a coaxial needle disclosed herein can be configured to have at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 21, at most 22, at most 23, at most 24, at most 25, at most 50, at most 75, at most 100, at most 125, at most 150, at most 200, at most 250, at most 300, at most 350, at most 400, at most 450, at most 500, at most 600, at most 700, at most 800, at most 900, or at most 1,000 paired openings. In some embodiments, a coaxial needle disclosed herein can be configured to have between 1-1,000, 1-500, 1-250, 1-100, 1-50, 1-25, 25-1,000, 25-500, 25-250, 25-100, 25-50, 50-1,000, 50-500, 50-250, 50-100, 100-1,000, 100-500, 100-250, 250-1,000, 250-500, or 500-1,000 paired openings.

The area of a paired opening is often a function of the size, shape, and arrangement of side wall openings. The side wall openings that form a paired opening can be the same, or different, sizes. The area of a paired opening can generally be as large as the area of the smallest component side wall opening. In some embodiments, a paired opening of the disclosure has an area of 0.1 mm² to 80 mm². In some embodiments a paired opening of the disclosure has an area of 0.1 mm² to 0.5 mm², 0.1 mm² to 1 mm², 0.1 mm² to 5 mm², 0.1 mm² to 10 mm², 0.1 mm² to 20 mm², 0.1 mm² to 30 mm², 0.1 mm² to 40 mm², 0.1 mm² to 50 mm², 0.1 mm² to 60 mm², 0.1 mm² to 70 mm², 0.1 mm² to 80 mm², 0.5 mm² to 1 mm², 0.5 mm² to 5 mm², 0.5 mm² to 10 mm², 0.5 mm² to 20 mm², 0.5 mm² to 30 mm², 0.5 mm² to 40 mm², 0.5 mm² to 50 mm², 0.5 mm² to 60 mm², 0.5 mm² to 70 mm², 0.5 mm² to 80 mm², 1 mm² to 5 mm², 1 mm² to 10 mm², 1 mm² to 20 mm², 1 mm² to 30 mm², 1 mm² to 40 mm², 1 mm² to 50 mm², 1 mm² to 60 mm², 1 mm² to 70 mm², 1 mm² to 80 mm², 5 mm² to 10 mm², 5 mm² to 20 mm², 5 mm² to 30 mm², 5 mm² to 40 mm², 5 mm² to 50 mm², 5 mm² to 60 mm², 5 mm² to 70 mm², 5 mm² to 80 mm², 10 mm² to 20 mm², 10 mm² to 30 mm², 10 mm² to 40 mm², 10 mm² to 50 mm², 10 mm² to 60 mm², 10 mm² to 70 mm², 10 mm² to 80 mm², 20 mm² to 30 mm², 20 mm² to 40 mm², 20 mm² to 50 mm², 20 mm² to 60 mm², 20 mm² to 70 mm², 20 mm² to 80² mm, 30 mm² to 40² mm, 30 mm² to 50 mm², 30 mm² to 60 mm², 30 mm² to 70 mm², 30 mm² to 80 mm², 40 mm² to 50 mm², 40 mm² to 60 mm², 40 mm² to 70 mm², 40 mm² to 80 mm², 50 mm² to 60 mm², 50 mm² to 70 mm², 50 mm² to 80 mm², 60 mm² to 70 mm², 60 mm² to 80 mm², or 70 mm² to 80 mm². In some embodiments a paired opening of the disclosure has an area of 0.1 mm², 0.5 mm², 1 mm², 5 mm², 10 mm², 20 mm², 30 mm², 40 mm², 50 mm², 60 mm², 70 mm², or 80 mm². In some embodiments a paired opening of the disclosure has an area of at least 0.1 mm², at least 0.5 mm², at least 1 mm², at least 5 mm², at least 10 mm², at least 20 mm², at least 30 mm², at least 40 mm², at least 50 mm², at least 60 mm², or at least 70 mm². In some embodiments a paired opening of the disclosure has an area of at most 0.5 mm², at most 1 mm², at most 5 mm², at most 10 mm², at most 20 mm², at most 30 mm², at most 40 mm², at most 50 mm², at most 60 mm², at most 70 mm², or at most 80 mm².

D. Expanding Member

In some embodiments, the coaxial needle comprises an expanding member. Expanding members can increase or decrease in size depending on the condition (e.g., temperature, rigidity, pressure, etc.) of the surrounding environment. For example, an expanding member can experience a temperature change following injection into a tissue and expand to provide an increased volume into which fluid can be injected. In some embodiments, an expanding member of the disclosure is composed of or comprises of a shape memorial alloy such as nitinol. In some embodiments, the inner elongate tubular body, outer elongate tubular body, or both the inner and outer elongate tubular body are composed of or comprise a metallic material comprising a shape memory alloy such as nitinol.

In some embodiments, the expanding member is located on or adjacent to the distal end of a coaxial needle disclosed herein. In some embodiments, the ratio of nitinol is between 0.8:1 and 1.2:1. In some embodiments, the nickel-titanium ratio of nitinol is about 0.8, about 0.81, about 0.82, about 0.83, about 0.84, about 0.85, about 0.86, about 0.87, about 0.88, about 0.89, about 0.9, about 0.91, about 0.92, about 0.93, about 0.94, about 0.95, about 0.96, about 0.97, about 0.98, about 0.99, about 1, about 1.01, about 1.02, about 1.03, about 1.04, about 1.05, about 1.06, about 1.07, about 1.08, about 1.09, about 1.1, about 1.11, about 1.12, about 1.13, about 1.14, about 1.15, about 1.16, about 1.17, about 1.18, about 1.19, or about 1.2.

E. Needle Dimensions

Tubular body design considerations: The coaxial needles can comprise inner and outer elongate tubular bodies of various dimensions, which can include the length of the body and diameter of the lumen. The dimensions of a coaxial needle can be selected based on design considerations including, for example, mechanical strength of the needle, surface stiffness of the intended substrate, location of the intended injection site, composition of the injected fluid, and identity of the intended substrate (e.g. a biological tissue). Compared to an outer elongate tubular body, an inner elongate tubular body has a smaller diameter so that it can fit inside the outer elongate tubular body. Inner and outer elongate tubular bodies can be of the same length or of different lengths. The inner elongate tubular body of a coaxial needle can be longer or shorter than the outer elongate tubular body of the coaxial needle.

Inner tubular body diameter: In some instances, the diameter of the lumen of an inner elongate tubular body is expressed using the Birmingham gauge system. In some embodiments, the coaxial needle has an inner elongate tubular body with a lumen diameter of 7 gauge, 8 gauge, 9 gauge, 10 gauge, 11 gauge, 12 gauge, 13 gauge, 14 gauge, 15 gauge, 16 gauge, 17 gauge, 18 gauge, 19 gauge, 20 gauge, 21 gauge, 22 gauge, 22s gauge, 23 gauge, 24 gauge, 25 gauge, 26 gauge, 26s gauge, 27 gauge, 28 gauge, 29 gauge, 30 gauge, 31 gauge, 32 gauge, or 33 gauge.

In some instances, the diameter of a lumen formed by an elongate tubular body is expressed via the metric system. In some embodiments, a coaxial needle of the disclosure has an inner elongate tubular body with a lumen diameter of between 0.1 mm to 400 mm. In some embodiments, a coaxial needle of the disclosure has an inner elongate tubular body with a lumen diameter of between 0.1 mm to 0.5 mm, between 0.1 mm to 1 mm, between 0.1 mm to 10 mm, between 0.1 mm to 20 mm, between 0.1 mm to 30 mm, between 0.1 mm to 40 mm, between 0.1 mm to 50 mm, between 0.1 mm to 100 mm, between 0.1 mm to 200 mm, between 0.1 mm to 300 mm, between 0.1 mm to 400 mm, between 0.5 mm to 1 mm, between 0.5 mm to 10 mm, between 0.5 mm to 20 mm, between 0.5 mm to 30 mm, between 0.5 mm to 40 mm, between 0.5 mm to 50 mm, between 0.5 mm to 100 mm, between 0.5 mm to 200 mm, between 0.5 mm to 300 mm, between 0.5 mm to 400 mm, between 1 mm to 10 mm, between 1 mm to 20 mm, between 1 mm to 30 mm, between 1 mm to 40 mm, between 1 mm to 50 mm, between 1 mm to 100 mm, between 1 mm to 200 mm, between 1 mm to 300 mm, between 1 mm to 400 mm, between 10 mm to 20 mm, between 10 mm to 30 mm, between 10 mm to 40 mm, between 10 mm to 50 mm, between 10 mm to 100 mm, between 10 mm to 200 mm, between 10 mm to 300 mm, between 10 mm to 400 mm, between 20 mm to 30 mm, between 20 mm to 40 mm, between 20 mm to 50 mm, between 20 mm to 100 mm, between 20 mm to 200 mm, between 20 mm to 300 mm, between 20 mm to 400 mm, between 30 mm to 40 mm, between 30 mm to 50 mm, between 30 mm to 100 mm, between 30 mm to 200 mm, between 30 mm to 300 mm, between 30 mm to 400 mm, between 40 mm to 50 mm, between 40 mm to 100 mm, between 40 mm to 200 mm, between 40 mm to 300 mm, between 40 mm to 400 mm, between 50 mm to 100 mm, between 50 mm to 200 mm, between 50 mm to 300 mm, between 50 mm to 400 mm, between 100 mm to 200 mm, between 100 mm to 300 mm, between 100 mm to 400 mm, between 200 mm to 300 mm, between 200 mm to 400 mm, or between 300 mm to 400 mm.

In some embodiments, the coaxial needle has an inner elongate tubular body with a lumen diameter of 0.1 mm, 0.5 mm, 1 mm, 10 mm, 20 mm, 30 mm, 40 mm, 50 mm, 100 mm, 200 mm, 300 mm, or 400 mm. In some embodiments, the coaxial needle has an inner elongate tubular body with a lumen diameter of at least 0.1 mm, at least 0.5 mm, at least 1 mm, at least 10 mm, at least 20 mm, at least 30 mm, at least 40 mm, at least 50 mm, at least 100 mm, at least 200 mm, or at least 300 mm. In some embodiments a coaxial needle of the disclosure has an inner elongate tubular body with a lumen diameter of at most 0.5 mm, at most 1 mm, at most 10 mm, at most 20 mm, at most 30 mm, at most 40 mm, at most 50 mm, at most 100 mm, at most 200 mm, at most 300 mm, or at most 400 mm.

Inner tubular body length: In some embodiments, a coaxial needle of the disclosure comprises an inner elongate tubular body with a length of 0.5 cm to 200 cm. In some embodiments, a coaxial needle of the disclosure comprises an inner elongate tubular body with a length of 0.5 cm to 1 cm, 0.5 cm to 5 cm, 0.5 cm to 10 cm, 0.5 cm to 20 cm, 0.5 cm to 40 cm, 0.5 cm to 60 cm, 0.5 cm to 80 cm, 0.5 cm to 100 cm, 0.5 cm to 150 cm, 0.5 cm to 200 cm, 1 cm to 5 cm, 1 cm to 10 cm, 1 cm to 20 cm, 1 cm to 40 cm, 1 cm to 60 cm, 1 cm to 80 cm, 1 cm to 100 cm, 1 cm to 150 cm, 1 cm to 200 cm, 5 cm to 10 cm, 5 cm to 20 cm, 5 cm to 40 cm, 5 cm to 60 cm, 5 cm to 80 cm, 5 cm to 100 cm, 5 cm to 150 cm, 5 cm to 200 cm, 10 cm to 20 cm, 10 cm to 40 cm, 10 cm to 60 cm, 10 cm to 80 cm, 10 cm to 100 cm, 10 cm to 150 cm, 10 cm to 200 cm, 20 cm to 40 cm, 20 cm to 60 cm, 20 cm to 80 cm, 20 cm to 100 cm, 20 cm to 150 cm, 20 cm to 200 cm, 40 cm to 60 cm, 40 cm to 80 cm, 40 cm to 100 cm, 40 cm to 150 cm, 40 cm to 200 cm, 60 cm to 80 cm, 60 cm to 100 cm, 60 cm to 150 cm, 60 cm to 200 cm, 80 cm to 100 cm, 80 cm to 150 cm, 80 cm to 200 cm, 100 cm to 150 cm, 100 cm to 200 cm, or 150 cm to 200 cm. In some embodiments, a coaxial needle of the disclosure comprises an inner elongate tubular body with a length of 0.5 cm, 1 cm, 5 cm, 10 cm, 20 cm, 40 cm, 60 cm, 80 cm, 100 cm, 150 cm, or 200 cm. In some embodiments, a coaxial needle of the disclosure comprises an inner elongate tubular body with a length of at least 0.5 cm, at least 1 cm, at least 5 cm, at least 10 cm, at least 20 cm, at least 40 cm, at least 60 cm, at least 80 cm, at least 100 cm, or at least 150 cm. In some embodiments, a coaxial needle of the disclosure comprises an inner elongate tubular body with a length of at most 1 cm, at most 5 cm, at most 10 cm, at most 20 cm, at most 40 cm, at most 60 cm, at most 80 cm, at most 100 cm, at most 150 cm, or at most 200 cm.

Outer tubular body diameter: In some embodiments, a coaxial needle of the disclosure has an outer elongate tubular body with a lumen diameter of 8 gauge, 9 gauge, 10 gauge, 11 gauge, 12 gauge, 13 gauge, 14 gauge, 15 gauge, 16 gauge, 17 gauge, 18 gauge, 19 gauge, 20 gauge, 21 gauge, 22 gauge, 22s gauge, 23 gauge, 24 gauge, 25 gauge, 26 gauge, 26 gauge, 27 gauge, 28 gauge, 29 gauge, 30 gauge, 31 gauge, 32 gauge, 33 gauge, or 34 gauge. In some embodiments, a coaxial needle of the disclosure has an outer elongate tubular body with a lumen diameter of 0.5 mm to 500 mm. In some embodiments, a coaxial needle of the disclosure has an inner elongate tubular body with a lumen diameter of 0.5 mm to 1 mm, 0.5 mm to 10 mm, 0.5 mm to 20 mm, 0.5 mm to 30 mm, 0.5 mm to 40 mm, 0.5 mm to 50 mm, 0.5 mm to 100 mm, 0.5 mm to 200 mm, 0.5 mm to 300 mm, 0.5 mm to 400 mm, 0.5 mm to 500 mm, 1 mm to 10 mm, 1 mm to 20 mm, 1 mm to 30 mm, 1 mm to 40 mm, 1 mm to 50 mm, 1 mm to 100 mm, 1 mm to 200 mm, 1 mm to 300 mm, 1 mm to 400 mm, 1 mm to 500 mm, 10 mm to 20 mm, 10 mm to 30 mm, 10 mm to 40 mm, 10 mm to 50 mm, 10 mm to 100 mm, 10 mm to 200 mm, 10 mm to 300 mm, 10 mm to 400 mm, 10 mm to 500 mm, 20 mm to 30 mm, 20 mm to 40 mm, 20 mm to 50 mm, 20 mm to 100 mm, 20 mm to 200 mm, 20 mm to 300 mm, 20 mm to 400 mm, 20 mm to 500 mm, 30 mm to 40 mm, 30 mm to 50 mm, 30 mm to 100 mm, 30 mm to 200 mm, 30 mm to 300 mm, 30 mm to 400 mm, 30 mm to 500 mm, 40 mm to 50 mm, 40 mm to 100 mm, 40 mm to 200 mm, 40 mm to 300 mm, 40 mm to 400 mm, 40 mm to 500 mm, 50 mm to 100 mm, 50 mm to 200 mm, 50 mm to 300 mm, 50 mm to 400 mm, 50 mm to 500 mm, 100 mm to 200 mm, 100 mm to 300 mm, 100 mm to 400 mm, 100 mm to 500 mm, 200 mm to 300 mm, 200 mm to 400 mm, 200 mm to 500 mm, 300 mm to 400 mm, 300 mm to 500 mm, or 400 mm to 500 mm.

In some embodiments, a coaxial needle of the disclosure has an outer elongate tubular body with a lumen diameter of 0.5 mm, 1 mm, 10 mm, 20 mm, 30 mm, 40 mm, 50 mm, 100 mm, 200 mm, 300 mm, 400 mm, or 500 mm. In some embodiments, a coaxial needle of the disclosure has an inner elongate tubular body with a lumen diameter of at least 0.5 mm, at least 1 mm, at least 10 mm, at least 20 mm, at least 30 mm, at least 40 mm, at least 50 mm, at least 100 mm, at least 200 mm, at least 300 mm, or at least 400 mm. In some embodiments, a coaxial needle of the disclosure has an inner elongate tubular body with a diameter of at most 1 mm, at most 10 mm, at most 20 mm, at most 30 mm, at most 40 mm, at most 50 mm, at most 100 mm, at most 200 mm, at most 300 mm, at most 400 mm, or at most 500 mm.

Outer tubular body length: In some embodiments, a coaxial needle of the disclosure comprises an outer elongate tubular body with a length of 0.5 cm to 200 cm. In some embodiments, a coaxial needle of the disclosure comprises an outer elongate tubular body with a length of 0.5 cm to 1 cm, 0.5 cm to 5 cm, 0.5 cm to 10 cm, 0.5 cm to 20 cm, 0.5 cm to 40 cm, 0.5 cm to 60 cm, 0.5 cm to 80 cm, 0.5 cm to 100 cm, 0.5 cm to 150 cm, 0.5 cm to 200 cm, 1 cm to 5 cm, 1 cm to 10 cm, 1 cm to 20 cm, 1 cm to 40 cm, 1 cm to 60 cm, 1 cm to 80 cm, 1 cm to 100 cm, 1 cm to 150 cm, 1 cm to 200 cm, 5 cm to 10 cm, 5 cm to 20 cm, 5 cm to 40 cm, 5 cm to 60 cm, 5 cm to 80 cm, 5 cm to 100 cm, 5 cm to 150 cm, 5 cm to 200 cm, 10 cm to 20 cm, 10 cm to 40 cm, 10 cm to 60 cm, 10 cm to 80 cm, 10 cm to 100 cm, 10 cm to 150 cm, 10 cm to 200 cm, 20 cm to 40 cm, 20 cm to 60 cm, 20 cm to 80 cm, 20 cm to 100 cm, 20 cm to 150 cm, 20 cm to 200 cm, 40 cm to 60 cm, 40 cm to 80 cm, 40 cm to 100 cm, 40 cm to 150 cm, 40 cm to 200 cm, 60 cm to 80 cm, 60 cm to 100 cm, 60 cm to 150 cm, 60 cm to 200 cm, 80 cm to 100 cm, 80 cm to 150 cm, 80 cm to 200 cm, 100 cm to 150 cm, 100 cm to 200 cm, or 150 cm to 200 cm. In some embodiments, a coaxial needle of the disclosure comprises an outer elongate tubular body with a length of 0.5 cm, 1 cm, 5 cm, 10 cm, 20 cm, 40 cm, 60 cm, 80 cm, 100 cm, 150 cm, or 200 cm. In some embodiments, a coaxial needle of the disclosure comprises an outer elongate tubular body with a length of at least 0.5 cm, at least 1 cm, at least 5 cm, at least 10 cm, at least 20 cm, at least 40 cm, at least 60 cm, at least 80 cm, at least 100 cm, or at least 150 cm. In some embodiments, a coaxial needle of the disclosure comprises an outer elongate tubular body with a length of at most 1 cm, at most 5 cm, at most 10 cm, at most 20 cm, at most 40 cm, at most 60 cm, at most 80 cm, at most 100 cm, at most 150 cm, or at most 200 cm.

### II. Fluid Injection Systems

The coaxial needles of the disclosure can be incorporated into injection systems. In some instances, the devices can be fluidically connected to a reservoir. Fluid flow can be directed from a reservoir through the coaxial needle and into a substrate. A coaxial needle can be connected to a reservoir directly or indirectly. For example, a reservoir containing the fluid to be injected can be connected to a coaxial needle described herein by a connecting a vessel such as a pipe or tube. In some embodiments, the coaxial needle can be connected directly to a reservoir such as a syringe.

Fluid injection systems can control, for example, fluid flow rate through a coaxial needle and the location of fluid delivery. Fluid flow rate can be adjusted by, for example, controlling the rate of fluid flow from a reservoir into a coaxial needle. In some embodiments, a fluid injection system described herein can comprise one or more flow modulation components. Non-limiting examples of flow modulation components include, pumps (e.g. pneumatic pumps or peristaltic pumps), vacuums, pressure chambers, pistons, plungers, and valves such as ball valves, butterfly valves, choke valves, diaphragms, globe valves, gate valves, knife valves, needle valves, pinch valves, piston valves, plug valves, solenoid valves, and spool valves. Fluid injection systems and flow modulation components disclosed herein can be controlled manually or electronically. In some embodiments, fluid flow is driven by a pneumatic force or by a force provided by a user on a plunger. In some embodiments, a fluid injection system or flow modulation component is controlled by a computer system in communication with one or more flow modulation components.

Additionally, a fluid injection system can adjust the flow rate through a coaxial needle via the movement of one or more elongate tubular bodies. Movement of elongate tubular bodies can modulate the number and size of paired openings present on a coaxial needle allowing for control of flow rate and the depth of fluid injection. The movement of elongate tubular bodies can be controlled manually or electronically. In some embodiments, the movement of one or more elongate tubular bodies can be controlled via a computer system in communication with one or more of the elongate tubular bodies. In some embodiments, the movement of one or more elongate tubular bodies can be controlled manually via a handle connected to an elongate tubular body. A handle connected to an elongate tubular body can allow, for example, for manual control of the movement of a tubular body in the axial or radial direction.

In some embodiments, a fluid injection system can control the location of a coaxial needle in relation to the substrate to be injected (injection substrate). For example, a fluid injection system can control the movement of a coaxial needle in the X, Y, or Z (injection depth) direction. In some embodiments, a computer controls the movement of a coaxial needle in relation to the injection substrate.

### III. Methods of Use

A. Repairing Damaged Tissue

Devices and systems disclosed herein can be used to introduce material, including fluid and particles, into substrates. In some embodiments the material comprises cells. Examples of substrates include, without limitation, biological tissue and tissue grafts. In some instances, devices and systems disclosed herein can be used in a method to aid in the repair of damaged tissues. In some instances, the method comprises introducing cells into a substrate. For example, the devices and systems can promote the healing of cartilage tissue by facilitating the cellularization of cartilage with chondrocytes, the predominant cell type in cartilage. Chondrocytes that are lost due to injury, disease, age, or other factors are often not replaced with viable cells due to the avascular nature of cartilage. Additionally, cells have difficulty migrating through dense cartilage. Thus, cell death causes cartilage tissue to become frail and start to fragment and fray. Recellularization of cartilage tissue aids in the tissue repair process.

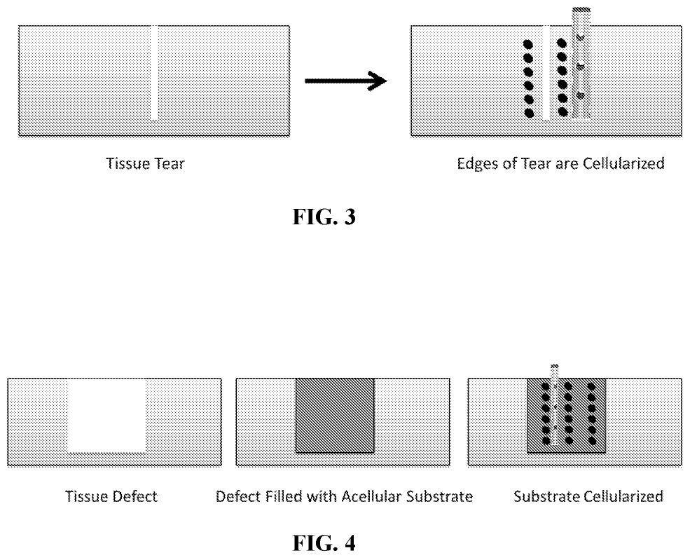

In some embodiments, a method of the disclosure directly recellularizes cartilage tissue by injecting cells around the edges of a tissue tear or defect as shown in FIG. 3. In some embodiments, a substrate (e.g., a tissue graft) is implanted into a tissue defect and then cellularized with a method of the disclosure as shown in FIG. 4. Tissue grafts injected with a method of the disclosure can be, for example, allogenic tissue grafts (allograft) or autologous tissue grafts. Advantages of an autologous tissue graft include a reduction of immunogenicity concerns, while allogenic grafts possess the advantage of broader availability. In some embodiments, a substrate is injected with cells and then implanted into a subject. A method disclosed herein can also include culturing or expanding the cells in an injected substrate prior to implantation into a subject. In some embodiments, a substrate such as a decellularized allogenic graft is implanted into a tissue defect before being injected with cells via a method of the disclosure. In some embodiments, a decellularized allogenic graft can be irradiated and/or sterilized prior to implantation into a subject.

In some embodiments, an allogenic graft is decellularized prior to injection using a coaxial needle disclosed herein. Decellularization of an allogenic graft can reduce immunogenicity. Injection of an implanted decellularized allogenic graft with cells can facilitate graft incorporation and stimulate healing. In some embodiments, a method of the disclosure aids in tissue repair by injecting fluid comprising cells, a growth factor, an adhesion molecule, a component of extracellular matrix (ECM), a synthetic polymer, a natural polymer, a biochemical factor, a protein, an enzyme, a therapeutic agent, a cross-linking agent, a photoinitiator, an additional agent, or any combination thereof into a substrate.

In some embodiments, a method of the disclosure mitigates the effects of compressive forces exerted by a substrate on injected fluid. By delivering fluid through paired openings along the side wall of a coaxial needle, injection can take place at multiple substrate locations without the need to move or remove the needle. The presence of the needle within the substrate during injection at multiple locations can mitigate the effect of compressive forces exerted by the substrate on the fluid. By mitigating the effects of compressive forces, a method of the disclosure can facilitate the delivery of injected fluid into a substrate by preventing the expulsion of the fluid from the substrate by the compressive forces. Mitigation of compressive forces can facilitate the effective delivery of cells into a substrate.

B. Control of Fluid Flow

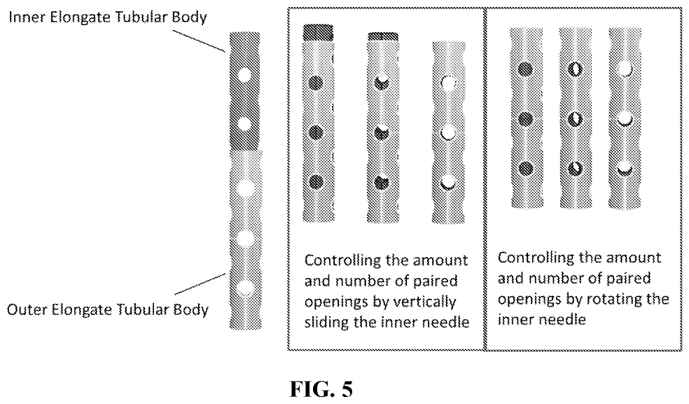

A method disclosed herein can comprise controlling the flow of fluid through a coaxial needle of the disclosure. For example, a user can control the flow rate of fluid through the coaxial needle or the location(s) at which fluid exits the needle. Control of fluid flow through the coaxial needle can be modulated by changing the configuration of elongate tubular bodies. The method can comprise controlling the position of an outer elongate tubular body relative to an inner elongate tubular body or vice versa. In some aspects, the method comprises controlling the position of elongate tubular bodies by moving one elongate tubular body relative to another elongate tubular body. The relative movement of one elongate tubular body with respect to another can change the number, size, and arrangement of paired openings located along the cylindrical side wall of the coaxial needle. An example of this is shown in FIG. 5. Each of the number, size, and arrangement of paired openings can affect the flow rate of fluid through the paired openings of a coaxial needle and the location of fluid injection within a substrate. In some embodiments, the method comprises moving the inner elongate tubular body, the outer elongate tubular body, or both the inner and outer elongate tubular body of the coaxial needle. The method of the disclosure can comprise moving the inner and/or outer elongate tubular body in an axial direction. Additionally or alternatively, the method can comprise rotating the inner and/or outer elongate tubular body about the longitudinal axes of the inner or outer tubular body (i.e. radial movement).

In some embodiments, the movement of elongate tubular bodies is controlled by a flow injection system comprising a computer. For example, a user can input an arrangement of paired openings to be used for injection. The user can then place the coaxial needle in the desired substrate and direct the computer to proceed with injection. The flow injection system can control various parts and functions of the coaxial needle through actuators and pumps. The flow injection system can then adjust the movement of the elongate tubular bodies of the coaxial needle to provide the arrangement of paired openings. For example, an actuator can control the position and movement of the inner elongate tubular body, the outer elongate tubular body, or both the inner and outer elongate tubular body of the coaxial needle in the axial or radial direction. The arrangement of paired openings can dictate the fluid flow rate through the coaxial needle as well as the location of fluid injection within the substrate. In some embodiments, fluid flows through an opening on the distal end of the coaxial needle.

In some embodiments, fluid flow through a coaxial needle is controlled via a fluid injection system of the disclosure. Fluid flow rate can be adjusted by controlling a flow modulation component disclosed herein. For example, a user can increase the flow rate through a coaxial needle by increasing the amount of force exerted on a plunger that drives the fluid from a reservoir into the coaxial needle. In some embodiments, the flow rate of fluid is modulated by altering the configuration of one or more valves present in a fluid injection system. In some embodiments, fluid flow rate is controlled by a pump present in a fluid injection system. A user can control fluid injection systems and flow modulation components disclosed herein manually or electronically. For example, flow rate through a fluid injection system can be detected and automatically adjusted by a computer system.

In some embodiments, a method disclosed herein comprises discharging fluid from a coaxial needle in multiple pulses. In some instances, each pulse can deliver the same or a different cell type to a substrate. For example, a needle can be inserted into a substrate and a first paired opening of interest can be formed by moving an inner or outer elongate tubular body. A fluid comprising a first cell type can then be discharged through the needle to enter the substrate. In some instances, a second paired opening can be formed and/or the first paired opening closed. The second paired opening can be at a different location than the first paired opening. In some cases, the first fluid comprising the first cell type can be discharged through the second paired opening. Alternatively or in addition, a second cell type can be discharged through the second paired opening and into the substrate. In some instances, flowing one or more fluids through multiple paired openings through a method of the disclosure can deliver cells to multiple locations in a substrate while decreasing the amount of times a needle pierces the substrate. In some instances, the position of the coaxial needle can be changed relative to the substrate when opening and closing paired openings. For example, paired openings can be opened or closed while the depth of a needle within an injection site is changed to cause the discharge of fluid at different locations in the substrate. Additionally or alternatively, by changing the arrangement of paired openings fluid can be discharged at different positions around the circumference of a needle without having to remove the needle from the substrate. In some instances, the position of the coaxial needle does not change relative to the substrate when opening and closing paired openings.

The process of the preceding paragraph can be repeated. In some instances, a different cell type is injected each time the process is repeated. In some instances, the same cell type is injected each time the process is repeated. For example, the process can be repeated 1, 2, 3, 4, 5, 6, 7, 8, 9, 10, 11, 12, 13, 14, 15, or more times with the same or different fluid or cell type being injected at each repetition. In some instances, the needle can be removed from the substrate and inserted into a different location of the substrate prior to discharging a fluid into the substrate.

C. Utilization of Shape Memory Alloys

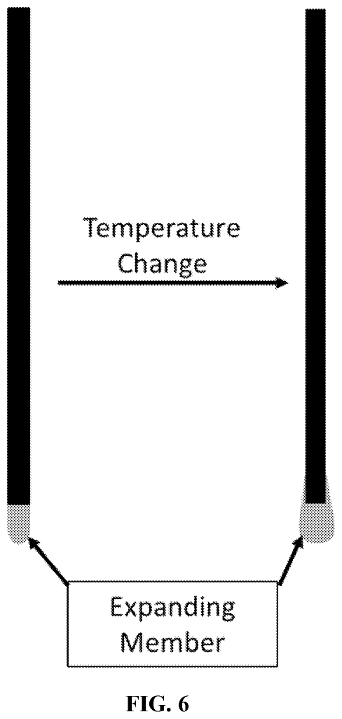

In some embodiments, a method of the disclosure comprises injecting a substrate with a coaxial needle comprising an expanding member to facilitate the delivery of fluid. For example, a coaxial needle can be inserted into a substrate with an expanding member at an initial temperature. The initial temperature of the expanding member can be set so that insertion of the needle into the substrate causes the expanding member to increase in size due to a temperature change. In some examples, the method includes expansion of the substrate. Expansion of the substrate can be due to the increase in size of the expanding member as seen in FIG. 6. Expanding the substrate can mitigate the effects of compressive forces from the substrates and facilitates the delivery of fluid via the coaxial needle. Expansion of an expanding member on the outer tubular body can also be used to increase the coefficient of friction between the outer member and the substrate. In some embodiments, the method comprises increasing the temperature of the expanding member (e.g., at or above a pre-determined threshold deformation temperature) during or after insertion into a substrate. In some embodiments the method comprises decreasing the temperature of the expanding member (e.g., at or below a pre-determined threshold deformation temperature) upon insertion into a substrate.

In some embodiments, a method of the disclosure utilizes a coaxial needle with an inner elongate tubular body, an outer elongate tubular body, or both an inner and outer elongate tubular body made up of a metallic material comprising a shape memory alloy to deliver fluid to a location within a substrate that is not easily accessible with a straight needle. For example, a shape memory alloy coaxial needle at an initial temperature can be inserted into a substrate in a straightened orientation. Insertion of the needle into the substrate can cause a change in the temperature of the needle causing it to bend into a new shape as shown in FIG. 2. The change in shape of the needle can allow delivery of fluid to an area of the substrate that is different than the area that fluid would have been delivered to had the needle retained its original shape. Alternatively and/or additionally, the change in shape of the needle can allow for the controlled delivery of fluid into different locations in the substrate. For example, the method can comprise curving the needle at different directions (e.g., left, right, etc.) or angles (e.g., 90 degrees, 60 degrees, 30 degrees, or 10 degrees) relative to the orientation of the outer or inner tubular body as the tubular body penetrates the substrate, in order to inject cells in different locations within the substrate. In some instances, the method comprises deforming the needle so that it is curved at a predetermined angle (e.g., 90 degrees from the original shape) and, once inserted into the substrate, can be returned to the original shape. In some embodiments, fluid can be injected to a first location within the substrate (e.g. pointed by the 90-degree curved needle), and further injected to other locations (e.g., injected when the needle has a 60, 30, or 10-degree curve as the needle returns to the original shape). In some embodiments, fluid can be injected to a first location of the substrate and further injected to other locations when needle has a curve of between 0 degrees to 180 degrees, 0 degrees to 150 degrees, 0 degrees to 120 degrees, 0 degrees to 90 degrees, 0 degrees to 60 degrees, 0 degrees to 10 degrees, 10 degrees to 180 degrees, 10 degrees to 150 degrees, 10 degrees to 120 degrees, 10 degrees to 90 degrees, 10 degrees to 60 degrees, 10 degrees to 30 degrees, 30 degrees to 180 degrees, 30 degrees to 150 degrees, 30 degrees to 120 degrees, 30 degrees to 90 degrees, 30 degrees to 60 degrees, 60 degrees to 180 degrees, 60 degrees to 150 degrees, 60 degrees to 120 degrees, 60 degrees to 90 degrees, 90 degrees to 180 degrees, 90 degrees to 150 degrees, 90 degrees to 120 degrees, 120 degrees to 180 degrees, 120 degrees to 150 degrees, or 150 degrees to 180 degrees as the needle returns to the original shape. In some embodiments, the method comprises inducing an increase in the temperature of a shape memory alloy needle to cause a shape change. In some embodiments, the method comprises inducing a decrease in the temperature of a shape memory alloy needle to cause a shape change.

### IV. Composition of Injected Fluid

In some embodiments, a coaxial needle of the disclosure is used to inject fluid into a substrate such as biological tissue. In some embodiments, fluid is delivered to a coaxial needle of the disclosure via a reservoir that is fluidically connected to the coaxial needle. In some non-limiting examples, the fluid injected via a coaxial needle of the disclosure comprises a plurality of cells a growth factor, an adhesion molecule, a component of extracellular matrix (ECM), a synthetic polymer, a natural polymer, a biochemical factor, a protein, an enzyme, a therapeutic agent, a cross-linking agent, a photoinitiator, an additional agent, or a combination thereof.

Non-limiting examples of cells that, in some examples, are injected into a substrate via a coaxial needle of the disclosure include chondrogenic cells, chondrocytes, chondroprogenitor cells, chondrogenic precursors, keratinocytes, hair root cells, hair shaft cells, hair matrix cells, exocrine secretory epithelial cells, hormone secreting cells, epithelial cells, neural or sensory cells, photoreceptor cells, muscle cells, extracellular matrix cells, blood cells, cardiovascular cells, endothelial cells, vascular smooth muscle cells, kidney cells, pancreatic cells, immune cells, stem cells, germ cells, interstitial cells, stellate cells liver cells, gastrointestinal cells, lung cells, tracheal cells, vascular cells, skeletal muscle cells, cardiac cells, skin cells, smooth muscle cells, connective tissue cells, corneal cells, genitourinary cells, breast cells, reproductive cells, endothelial cells, epithelial cells, fibroblasts, Schwann cells, adipose cells, bone cells, bone marrow cells, cartilage cells, pericytes, mesothelial cells, cells derived from endocrine tissue, stromal cells, progenitor cells, lymph cells, endoderm-derived cells, ectoderm-derived cells, mesoderm-derived cells, pericytes, chondroblasts, mesenchymal stem cells, connective tissue fibroblasts, tendon fibroblasts, bone marrow reticular tissue fibroblasts, non-epithelial fibroblasts, pericytes, osteoprogenitor cells, osteoblasts, osteoclasts, articular chondrocytes, stem cells, progenitor cells, totipotent cells, pluripotent cells, multipotent cells, and induced pluripotent stem cells. In some embodiments, injected cells are derived from stem cells, progenitor cells, totipotent cells, pluripotent cells, multipotent cells, or induced pluripotent cells. In some embodiments, cells injected via a coaxial needle of the disclosure are genetically modified. Injected cells are, in some examples, injected as collections of cells that are adherent to one another to form a microspheroid or cell paste. In some embodiments, a combination of cells is injected. For example, a combination of mesenchymal stem cells and chondrocytes, or a combination of mesenchymal stem cells and osteoblasts can be injected.

In some embodiments, the cell density of a fluid of the disclosure is about 1 cell/pL, about 10 cells/pL, about 100 cells/pL, about 1 cell/nL, about 10 cells/nL, about 100 cells/nL, about 1 cell/μL, about 10 cells/μL, about 100 cells/μL, about 1000 cells/μL, about 10,000 cells cells/μL, about 100,000 cells/μL. In some embodiments, the cell density of the cell suspension is about 2×10⁶ cells/mL, about 3×10⁶ cells/mL, about 4×10⁶ cells/mL, about 5×10⁶ cells/mL, about 6×10⁶ cells/mL, about 7×10⁶ cells/mL, about 8×10⁶ cells/mL, about 9×10⁶ cells/mL, about 10×10⁶ cells/mL, about 15×10⁶ cells/mL, about 20×10⁶ cells/mL, about 25×10⁶ cells/mL, about 30×10⁶ cells/mL, about 35×10⁶ cells/mL, about 40×10⁶ cells/mL, about 45×10⁶ cells/mL, or about 50×10⁶ cells/mL. In further embodiments a fluid of the disclosure comprises one, or more than one cell type. For example, a fluid of the disclosure contains, in some embodiments, 1, 2, 3, 4, 5, 6, 7, 8, 9, 10, 12, 13, 14, 15, 16, 17, 18, 19, or 20 cell types. In some embodiments, the cell suspension comprises more than 20 cell types.

Cells injected via a coaxial needle of the disclosure are, in some examples, harvested, derived, or isolated from an autologous, allogenic, or xenogenic donor tissue. In some embodiments, the donor tissue is derived from a human, a monkey, an ape, a gorilla, a chimpanzee, a cow, a horse, a dog, a cat, a goat, a sheep, a pig, a rabbit, a chicken, a turkey, a guinea pig, a rat, or a mouse. In some instances, cells are obtained and cultured in vitro prior to injection. In some instances, cells are obtained and differentiated in vitro prior to injection.

In some embodiments, the cell suspension comprises a growth factor. In some embodiments, the growth factor is selected from adrenomedullin (AM), angiopoietin (Ang), autocrine motility factor, endothelin-1, platelet derived growth factor (PDGF), growth/differentiation factors such as GDF-1, GDF-5. GDF-9, and GDF-8; bone morphogenetic proteins (BMPs), brain-derived neurotrophic factor (BDNF), colony-stimulating factor (CSF), epidermal growth factor (EGF), erythropoietin (EPO), fibroblast growth factor (FGF), glial cell line-derived neurotrophic factor (GDNF), granulocyte colony-stimulating factor (G-CSF), granulocyte macrophage colony-stimulating factor (GM-CSF), growth differentiation factor-9 (GDF9), hepatocyte growth factor (HGF), hepatoma-derived growth factor (HDGF), insulin, insulin-like growth factor (IGF), migration-stimulating factor, myostatin (GDF-8), nerve growth factor (NGF) and other neurotrophins, platelet-derived growth factor (PDGF), thrombopoietin (TPO), transforming growth factor alpha (TGF-α), transforming growth factor beta (TGF-β), tumor necrosis factor-alpha (TNF-α), vascular endothelial growth factor (VEGF), placental growth factor (P1GF), fetal bovine somatotrophin, interleukins (IL), such as IL-1, IL-2, IL-3, IL-4, IL-5, IL-6, and IL-7; or a combination of any of the foregoing. In some embodiments, the cell suspension comprises TGF-β1 and FGF2.

In some embodiments, the injected fluid comprises a therapeutic agent. In some embodiments, the therapeutic agent is selected from an antibiotic and/or an antimycotic. In some embodiments, the antibiotic is penicillin, streptomycin, actinomycin D, ampicillin, blasticidin, carbenicillin, cefotaxime, fosmidomycin, gentamicin, kanamycin, neomycin, polymyxin B, or a combination thereof. In some embodiments, the antimycotic is amphotericin B, nystatin, natamycin or a combination thereof. In some embodiments, the therapeutic agent is selected from an anti-inflammatory therapeutic agent. In some embodiments, the anti-inflammatory therapeutic agent is a non-steroidal anti-inflammatory therapeutic agent. In some embodiments, the non-steroidal anti-inflammatory therapeutic agent is a cyclooxygenase (COX) inhibitor. In some embodiments, the COX inhibitor is selected from a COX1 inhibitor, COX2 inhibitor, or a combination thereof. In some embodiments, the anti-inflammatory therapeutic agent comprises a steroid. In some embodiments, the steroid is a glucocorticoid. In some embodiments, the glucocorticoid is dexamethasone.

### V. Injection Substrates

Non-limiting examples of substrates that, in some embodiments, are injected using a coaxial needle of the disclosure include an acellular or cellularized scaffold, such as an alginate scaffold, a collagen scaffold, a polyethylene glycol scaffold or a silk scaffold; or biological tissue such as orthopedic tissue, osteochondral tissue, chondral tissue, muscular tissue, connective tissue, epidermal tissue, intestinal tissue, neuronal tissue, reproductive tissue, pancreatic tissue, ocular tissue, breast tissue, cardiac tissue, osseous tissue, a ligament, a tendon, liver tissue, splenic tissue, and renal tissue. In some embodiments the scaffold is or comprises a hydrogel scaffold. In some embodiments, the scaffold is or comprises a fibrous scaffold. In some embodiments, a method disclosed herein includes the injection of devitalized tissue. For example, a method disclosed herein can inject a tissue allograft that has been decellularized, sterilized, and/or irradiated. Sterilizing, irradiating, and decellularizing an allograft can prevent an immune response when the allograft is implanted into the subject. In some embodiments, tissue injected with a coaxial needle of the disclosure is obtained from a human, a monkey, an ape, a gorilla, a chimpanzee, a cow, a horse, a dog, a cat, a goat, a sheep, a pig, a rabbit, a chicken, a turkey, a guinea pig, a rat, or a mouse. Substrates injected with a method disclosed herein are, in some examples, first implanted into a tissue defect as shown in FIG. 4. Alternatively and/or additionally, substrates injected with a method disclosed herein can be injected into a substrate prior to subsequent implantation of the substrate into a tissue defect. In some embodiments, the substrate injected with a method disclosed herein is a tissue containing a defect.

### EXEMPLARY EMBODIMENTS

Among the exemplary embodiments are:

Embodiment 1) A coaxial needle comprising: (a) a proximal end, (b) a distal end, and (c) a first elongate tubular body and a second elongate tubular body that are arranged concentrically, wherein the first elongate tubular body and the second elongate tubular body comprise: (i) a cylindrical side wall forming a lumen that extends along a longitudinal axis between the proximal end and the distal end, and (ii) a plurality of side wall openings arranged along the cylindrical side wall; wherein the second elongate tubular body is movable relative to the first elongate tubular body to form a plurality of configurations, wherein at least one of the plurality of configurations comprises a paired opening formed by an overlap of at least one of the plurality of side wall openings of the first elongate tubular body and at least one of the plurality of side wall openings of the second elongate tubular body.

Embodiment 2) The coaxial needle of embodiment 1, wherein the paired opening is formed by one of the plurality of side wall openings of the first elongate tubular body and one of the plurality of sidewall openings of the second elongate tubular body. Embodiment 3) The coaxial needle of embodiment 1 or 2, wherein the second elongate tubular body is arranged concentrically within the lumen of the first elongate tubular body. Embodiment 4) The coaxial needle of embodiment 3, wherein the lumen of the first elongate tubular body has a diameter of 0.5 mm to 500 mm. Embodiment 5) The coaxial needle of embodiment 3 or 4, wherein the lumen of the second elongate tubular body has a diameter of about 0.1 mm to about 400 mm. Embodiment 6) The coaxial needle of any one of embodiments 3-5, further comprising a piercing member located on the distal end and attached to the first elongate tubular body. Embodiment 7) The coaxial needle of any one of embodiments 3-5, further comprising a piercing member located on the distal end and attached to the second elongate tubular body. Embodiment 8) The coaxial needle of embodiment 1 or 2, wherein the first elongate tubular body is arranged concentrically within the lumen of the second elongate tubular body. Embodiment 9) The coaxial needle of embodiment 8, wherein the lumen of the first elongate tubular body has a diameter of 0.1 mm to 400 mm. Embodiment 10) The coaxial needle of embodiment 8 or 9, wherein the lumen of the second elongate tubular body has a diameter of 0.5 mm to 500 mm. Embodiment 11) The coaxial needle of any one of embodiments 8-10, further comprising a piercing member located on the distal end and attached to the first elongate tubular body. Embodiment 12) The coaxial needle of any one of embodiments 8-10, further comprising a piercing member located on the distal end and attached to the second elongate tubular body. Embodiment 13) The coaxial needle of any one of embodiments 1-12, wherein the plurality of side wall openings of the first elongate tubular body is arranged in a random, linear, spiral, radial, Fibonacci spiral, logarithmic spiral, Archimedean spiral, zig-zag, or checkerboard pattern. Embodiment 14) The coaxial needle of any one of embodiments 1-12, wherein the plurality of openings of the first elongate tubular body is arranged in a pattern, wherein the pattern replicates the spacing of cells in a biological tissue. Embodiment 15) The coaxial needle of any one of embodiments 1-14, wherein the plurality of side wall openings of the second elongate tubular body is arranged in a random, linear, spiral, radial, Fibonacci spiral, logarithmic spiral, Archimedean spiral, zig-zag, or checkerboard pattern. Embodiment 16) The coaxial needle of any one of embodiments 1-14, wherein the plurality of openings of the second elongate tubular body is arranged in a pattern, wherein the pattern replicates the spacing of cells in a biological tissue. Embodiment 17) The coaxial needle of any one of embodiments 1-16, wherein an area of the paired opening is at most 80 mm². Embodiment 18) The coaxial needle of any one of embodiments 1-17, wherein the first elongate tubular body has a length of 0.5 cm to 200 cm. Embodiment 19) The coaxial needle of any one of embodiments 1-18, wherein the second elongate tubular body has a length of 0.5 cm to 200 cm. Embodiment 20) The coaxial needle of any one of embodiments 1-19, wherein at least one of the plurality of side wall openings of the first elongate tubular body has an elliptical shape, a circular shape, a rectangular shape, a triangular shape, or a tear drop shape. Embodiment 21) The coaxial needle of any one of embodiments 1-20, wherein at least one of the plurality of side wall openings of the second elongate tubular body has an elliptical shape, a circular shape, a rectangular shape, a triangular shape, or a tear drop shape. Embodiment 22) The coaxial needle of any one of embodiments 1-21, wherein at least one of the plurality of side wall openings of the first elongate tubular body or at least one of the plurality of side wall openings of the second elongate tubular body has an area of 0.1 mm² to 80 mm². Embodiment 23) The coaxial needle of any one of embodiments 1-22, further comprising an opening located on the distal end. Embodiment 24) The coaxial needle of any one of embodiments 1-23, further comprising an expanding member located on or adjacent to the distal end of the coaxial needle, wherein the expanding member is a metallic material; and the metallic material comprises a shape memory alloy. Embodiment 25) The coaxial needle of any one of embodiments 1-23, wherein the cylindrical side wall of the first elongate tubular body and the cylindrical side wall of the second elongate tubular body are a metallic material, wherein the metallic material comprises a shape memory alloy. Embodiment 26) The coaxial needle of embodiment 24 or 25, wherein the shape memory alloy is nitinol. Embodiment 27) The coaxial needle of any one of embodiments 1-26, wherein the second elongate tubular body is movable in the axial direction. Embodiment 28) The coaxial needle of any one of embodiments 1-27, wherein the second elongate tubular body can rotate about the longitudinal axis of the second elongate tubular body.

Embodiment 29) A method of injecting fluid into a substrate comprising: (a) inserting a coaxial needle into the substrate, wherein the coaxial needle comprises: (I) a proximal end, (II) a distal end, and (III) an inner elongate tubular body and an outer elongate tubular body that are arranged concentrically, wherein the inner elongate tubular body and the outer elongate tubular body comprise: (i) a cylindrical side wall forming a lumen that extends along a longitudinal axis between the proximal end and the distal end, and (ii) a plurality of side wall openings arranged along the cylindrical side wall; wherein at least one of the inner or outer elongate tubular bodies is movable relative to the other to form a plurality of configurations, wherein at least one of the plurality of configurations comprises a paired opening formed by an overlap of at least one of the plurality of side wall openings of the inner elongate tubular body and at least one of the plurality of side wall openings of the outer elongate tubular body; and (b) flowing a fluid into the lumen of the inner elongate tubular body such that the fluid passes through the paired opening and into the substrate.

Embodiment 30) The method of embodiment 29, wherein the paired opening is formed by one of the plurality of side wall openings of the first elongate tubular body and one of the plurality of sidewall openings of the second elongate tubular body. Embodiment 31) The method of embodiment 29 or 30, wherein the inner elongate tubular body is movable relative to the outer elongate tubular body. Embodiment 32) The method of embodiment 31, further comprising controlling a position of the inner elongate tubular body relative to the outer elongate tubular body. Embodiment 33) The method of embodiment 31 or 32, wherein the inner elongate tubular body moves in the axial direction in relation to the outer elongate tubular body. Embodiment 34) The method of any one of embodiments 31-33, wherein the inner elongate tubular body rotates about the longitudinal axis of the inner elongate tubular body. Embodiment 35) The method of embodiment 29 or 30, wherein the outer elongate tubular body is movable relative to the inner elongate tubular body. Embodiment 36) The method of embodiment 35, further comprising controlling a position of the outer elongate tubular body relative to the inner elongate tubular body. Embodiment 37) The method of embodiment 35 or 36, wherein the outer elongate tubular body moves in the axial direction in relation to the inner elongate tubular body. Embodiment 38) The method of any one of embodiments 35-37, wherein the outer elongate tubular body rotates about the longitudinal axis of the outer elongate tubular body. Embodiment 39) The method of any one of embodiments 29-38, wherein the plurality of side wall openings of the inner elongate tubular body is arranged in random, linear, spiral, radial, Fibonacci spiral, logarithmic spiral, Archimedean spiral, zig-zag, or checkerboard pattern. Embodiment 40) The method of any one of embodiments 29-38, wherein the plurality of openings of the inner elongate tubular body is arranged in a pattern, wherein the pattern replicates the spacing of cells in a biological tissue. Embodiment 41) The method of any one of embodiments 29-40, wherein the plurality of side wall openings of the outer elongate tubular body is arranged in a random, linear, spiral, radial, Fibonacci spiral, logarithmic spiral, Archimedean spiral, zig-zag, or checkerboard pattern. Embodiment 42) The method of any one of embodiments 29-40, wherein the plurality of openings of the outer elongate tubular body are arranged in a pattern, wherein the pattern replicates the spacing of cells in a biological tissue. Embodiment 43) The method of any one of embodiments 29-42, wherein an area of the paired opening is at most 80 mm². Embodiment 44) The method of any one of embodiments 29-43, wherein the fluid comprises a plurality of cells. Embodiment 45) The method of embodiment 44, wherein the plurality of cells comprises chondrogenic cells, chondrogenic precursors, multipotent cells, or pluripotent cells. Embodiment 46) The method of any one of embodiments 29-45, wherein the fluid flows through an opening located on the distal end of the coaxial needle. Embodiment 47) The method of embodiment 46, wherein the opening located on the distal end of the coaxial needle increases in size after being inserted into the substrate. Embodiment 48) The method of any one of embodiments 29-47, wherein the coaxial needle changes shape after being inserted into the substrate. Embodiment 49) The method any one of embodiments 29-48, wherein the substrate is a biological tissue. Embodiment 50) The method of embodiment 49, wherein the biological tissue is human tissue, orthopedic tissue, cartilage, or devitalized tissue. Embodiment 51) The method of any one of embodiments 29-48, wherein the substrate is an acellular scaffold.

Embodiment 52) A method of repairing a damaged tissue comprising: (a) implanting an acellular tissue scaffold into the damaged tissue, (b) inserting a coaxial needle into the acellular tissue scaffold, wherein the coaxial needle comprises: (I) a proximal end, (II) a distal end, and (III) an inner elongate tubular body and an outer elongate tubular body that are arranged concentrically, wherein the inner elongate tubular body and the outer elongate tubular body comprise: (i) a cylindrical side wall forming a lumen that extends along a longitudinal axis between the proximal end and the distal end, and (ii) a plurality of side wall openings arranged along the cylindrical side wall; wherein at least one of the inner or outer elongate tubular bodies is movable relative to the other to form a plurality of configurations, wherein at least one of the plurality of configurations comprises a paired opening formed by an overlap of at least one of the plurality of side wall openings of the inner elongate tubular body and at least one of the plurality of side wall openings of the outer elongate tubular body; and (c) flowing a fluid into the lumen of the inner elongate tubular body such that the fluid passes through the paired opening and into the acellular tissue scaffold.

Embodiment 53) The method of embodiment 52, wherein the paired opening is formed by one of the plurality of side wall openings of the first elongate tubular body and one of the plurality of sidewall openings of the second elongate tubular body. Embodiment 54) The method of embodiment 52 or 53, wherein the inner elongate tubular body is movable relative to the outer elongate tubular body. Embodiment 55) The method of embodiment 54, further comprising controlling a position of the inner elongate tubular body relative to the outer elongate tubular body. Embodiment 56) The method of embodiment 54 or 55, wherein the inner elongate tubular body moves in the axial direction in relation to the outer elongate tubular body. Embodiment 57) The method of any one of embodiments 54-56, wherein the inner elongate tubular body rotates about the longitudinal axis of the inner elongate tubular body. Embodiment 58) The method of any one of embodiments 52-57, wherein the outer elongate tubular body is movable relative to the inner elongate tubular body. Embodiment 59) The method of embodiment 58, further comprising controlling a position of the outer elongate tubular body relative to the inner elongate tubular body. Embodiment 60) The method of embodiment 58 or 59, wherein the outer elongate tubular body moves in the axial direction in relation to the inner elongate tubular body. Embodiment 61) The method of any one of embodiments 58-60, wherein the outer elongate tubular body rotates about the longitudinal axis of the outer elongate tubular body. Embodiment 62) The method of any one of embodiments 52-61, wherein the plurality of side wall openings of the inner elongate tubular body is arranged in a random, linear, spiral, radial, Fibonacci spiral, logarithmic spiral, Archimedean spiral, zig-zag, or checkerboard pattern. Embodiment 63) The method of any one of embodiments 52-61, wherein the plurality of openings of the inner elongate tubular body is arranged in a pattern, wherein the pattern replicates the spacing of cells in a biological tissue. Embodiment 64) The method of any one of embodiments 52-63, wherein the plurality of side wall openings of the outer elongate tubular body is arranged in a random, linear, spiral, radial, Fibonacci spiral, logarithmic spiral, Archimedean spiral, zig-zag, or checkerboard pattern. Embodiment 65) The method of any one of embodiments 52-63, wherein the plurality of openings of the outer elongate tubular body are arranged in a pattern, wherein the pattern replicates the spacing of cells in a biological tissue. Embodiment 66) The method of any one of embodiments 52-65, wherein an area of the paired opening is at most 80 mm². Embodiment 67) The method of any one of embodiments 52-66, wherein the fluid comprises a plurality of cells. Embodiment 68) The method of embodiment 67, wherein the plurality of cells comprises chondrogenic cells, pluripotent cells, multipotent cells, or chondrogenic precursors. Embodiment 69) The method of any one of embodiments 52-68, wherein the fluid flows through an opening located on the distal end of the coaxial needle. Embodiment 70) The method of embodiment 69, wherein the opening located on the distal end of the coaxial needle increases in size after being inserted into the substrate. Embodiment 71) The method of any one of embodiments 52-70, wherein the coaxial needle changes shape after being inserted into the substrate. Embodiment 72) The method of any one of embodiments 52-71, wherein the acellular tissue scaffold is a devitalized tissue allograft.

### DEFINITIONS

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

As used herein, the term “about” a number refers to that number plus or minus 10% of that number. The term “about” a range refers to that range minus 10% of its lowest value and plus 10% of its greatest value.

The section headings used herein are for organizational purposes only and are not to be construed as limiting the subject matter described.

### EXAMPLES

The following examples are included for illustrative purposes only and are not intended to limit the scope of the disclosure.

### Example 1

### Preparing Chondrocytes for Injection

Chondrocytes are harvested and prepared for injection.

Specifically, chondrocytes are isolated from healthy human articular cartilage. Sterile scalpels are used to excise articular cartilage from femoral condyles and tibia plateaus under aseptic conditions. Harvested cartilage samples are minced and treated with pronase at 37° C. for 30 min with slow agitation. After removing the pronase solution, cells are washed twice with PBS. Washed samples are treated with collagenase P at 37° C. overnight or until the cartilage matrix is completely digested and cells are free in suspension. Clumps of cells are broken up by repeated aspiration of the suspension. The cell suspension is transferred to a sterile 50 mL conical polypropylene tube, passing through a 40-70 μm filter. The tubes are subjected to centrifugation for 5 minutes at 400 ×g to pellet cells. Following centrifugation, the resulting supernatant is aspirated and the pelleted cells are washed twice using PBS and once with DMEM/F12 containing 10% fetal calf serum (FCS). The cells are resuspended in DMEM/Ham's F-12 containing 10% FCS. The cells are then counted. The resuspended cells are diluted with DMEM/F12 containing 10% FCS to yield a 1×106 cells/mL cell suspension.

### Example 2

### Treatment of Torn Cartilage Via Chondrocyte Injection with a Standard Needle

Chondrocytes are injected in the proximity of a cartilage tear where they exude extracellular matrix and aid in the treatment of the tear.

A chondrocytes suspension is prepared as described in EXAMPLE 1. The prepared chondrocyte suspension is loaded into a syringe equipped with a standard beveled needle. The chondrocyte suspension is injected as a single bolus at a single depth into an area of the torn cartilage. The administered chondrocytes are confined to an area within close proximity to the injection site, limiting the benefits of newly produced extracellular matrix to a localized area. A second injection of the chondrocyte suspension is also performed. During the second injection, the needle is gradually moved upwards out of the tissue as the chondrocyte suspension is released at a constant flow rate. The tissue compresses to fill the void caused by the needle as the needle is gradually moved upwards. This causes chondrocytes injected via the second injection to be pushed out of the tissue once the needle is removed. As a result, the tissue retains only a fraction of the cells injected via the second injection.

### Example 3

### Treatment of Torn Cartilage Via Chondrocyte Injection with a Needle of the Disclosure

Chondrocytes are injected uniformly around the perimeter of a cartilage tear where they exude extracellular matrix and aid in the treatment of the tear.

Specifically, chondrocytes are isolated from healthy human articular cartilage as previously described in EXAMPLE 1. Following isolation, the harvested chondrocyte suspension is loaded into a syringe equipped with a coaxial needle with a plurality of side wall openings as shown in FIG. 1. The coaxial needle is inserted into the torn cartilage immediately adjacent to the tear with its inner and outer portions configured to prevent fluidic communication between the inner lumen of the coaxial needle and the surrounding tissue. Once inserted, the syringe plunger is pushed downward to cause the flow of fluid from the syringe into the inner lumen of the coaxial needle. Rotation of the inner portion of the coaxial needle, combined with the application of force to the syringe plunger, causes the controlled injection of the chondrocyte solution through the side wall openings and into the damaged cartilage. Injection of the chondrocyte solution through the side wall openings of the coaxial needle allows chondrocytes to be distributed evenly along the length of the tear. The coaxial needle is then removed from the cartilage and the injection process is repeated adjacent to the other sides of the cartilage tear. The repeated injections with the coaxial needle result in the even distribution of chondrocytes around the perimeter of the cartilage tear. Unlike in EXAMPLE 2, where the benefits of newly produced extracellular matrix are limited to a localized area, the injected chondrocytes of this example (EXAMPLE 3) produce extracellular matrix across the entire area of the tear, facilitating its repair.

### Example 4

### Treatment of Torn Cartilage Via Chondrocyte Injection with a Shape Memory Alloy Needle of the Disclosure

Chondrocytes are injected uniformly in the proximity of a cartilage tear located in an area that is inaccessible to a traditional needle.

Chondrocytes are isolated from healthy human articular cartilage as previously described in EXAMPLE 1. The prepared chondrocyte suspension is loaded into a syringe equipped with a nitinol coaxial needle with side wall openings as shown in FIG. 1. The needle is inserted into an area of cartilage adjacent to the cartilage tear that is inaccessible to a straightened needle. The nitinol coaxial needle remains inserted into the cartilage for a period of time to allow for the temperature of the nitinol to increase. As shown in FIG. 2, the increased temperature causes the needle to change shape, positioning it in the proximity of the tear's previously inaccessible perimeter. Chondrocytes are then injected uniformly around the cartilage tear. Newly injected chondrocytes produce extracellular matrix, thus facilitating tissue repair.

### Example 5

### Treatment of Torn Cartilage Via Chondrocyte Injection with an Expanding Shape Memory Alloy Needle of the Disclosure

An expanding shape memory alloy needle of the disclosure further facilitates cartilage repair by mitigating the effects of tissue compression.

Chondrocytes are isolated from healthy human articular cartilage as previously described in EXAMPLE 1. The prepared chondrocyte suspension is loaded into a syringe equipped with a nitinol coaxial needle with side wall openings as shown in FIG. 1. The nitinol coaxial needle is inserted into the torn cartilage immediately adjacent to the tear with its inner and outer portions configured to prevent fluidic communication between the inner lumen of the coaxial needle and the surrounding tissue. The needle remains inserted into the cartilage for a period of time to allow for the temperature of the needle to increase. The increased temperature causes the needle to expand as shown in FIG. 6, which facilitates expansion of the tissue. Following tissue expansion, the syringe plunger is pushed downward to cause the flow of fluid from the syringe into the inner lumen of the coaxial needle. Rotation of the inner portion of the coaxial needle, combined with the application of force to the syringe plunger, causes the controlled injection of the chondrocyte solution through the side wall openings and into the damaged cartilage. Tissue expansion caused by the expansion of the coaxial needle mitigates the effects of tissue compression upon removal of the needle and allows for the injected volume of chondrocyte solution to be increased. Newly injected chondrocytes produce extracellular matrix, thus facilitating tissue repair.

### Example 6

### Treatment of a Tissue Defect Using a Scaffold

A tissue defect is first filled with an acellular scaffold. Following scaffold engraftment, the scaffold is cellularized via uniform chondrocyte injection.

For example, an acellular scaffold such as Matrigel®, is applied to a tissue defect as shown in FIG. 4. A coaxial needle as shown in FIG. 1 is fluidically connected to a syringe containing a cell suspension prepared as described in EXAMPLE 1. The coaxial needle is inserted into the scaffold with its inner and outer portions configured to prevent fluidic communication between the inner lumen of the coaxial needle and the surrounding environment. Once inserted, the syringe plunger is pushed downward to cause the flow of fluid from the syringe into the inner lumen of the coaxial needle. Rotation of the inner portion of the coaxial needle, combined with the application of force to the syringe plunger, causes the controlled injection of the chondrocyte solution through the side wall openings and into the scaffold. Chondrocyte injection is repeated throughout the scaffold to ensure uniform population of the scaffold with chondrocytes. Chondrocytes then begin to produce extracellular matrix, which allows the newly cellularized scaffold to integrate with existing cartilage tissue.

### Example 7

### Treatment of a Tissue Defect with a Revitalized Tissue Allograft

A tissue allograft is harvested from a donor and decellularized. The decellularized allograft is then used to fill a tissue defect in a patient. A coaxial needle as shown in FIG. 1 is fluidically connected to a syringe containing mesenchymal stem cells harvested from the patient. The cell solution is then injected throughout the allograft in a manner that is analogous to the injection into the tissue scaffold described in EXAMPLE 6. Over time, mesenchymal stem cells differentiate into chondrocytes and produce extracellular matrix. The injected differentiated cells do not illicit an immune response in the patient due to their autologous origin.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A coaxial needle comprising:
(a) a proximal end;
(b) a distal end; and
(c) a first elongate tubular body and a second elongate tubular body that are arranged concentrically, wherein the first elongate tubular body and the second elongate tubular body comprise:
(i) a cylindrical side wall forming a lumen that extends along a longitudinal axis between the proximal end and the distal end; and
(ii) a plurality of side wall openings arranged along the cylindrical side wall;
wherein the second elongate tubular body is movable relative to the first elongate tubular body to form a plurality of configurations, wherein at least one of the plurality of configurations comprises a paired opening formed by an overlap of at least one of the plurality of side wall openings of the first elongate tubular body and at least one of the plurality of side wall openings of the second elongate tubular body.

2. The coaxial needle of claim 1, wherein the paired opening is formed by one of the plurality of side wall openings of the first elongate tubular body and one of the plurality of sidewall openings of the second elongate tubular body.

3. The coaxial needle of claim 1 or 2, wherein the second elongate tubular body is arranged concentrically within the lumen of the first elongate tubular body.

4. The coaxial needle of claim 3, wherein the lumen of the first elongate tubular body has a diameter of 0.5 mm to 500 mm.

5. The coaxial needle of claim 3 or 4, wherein the lumen of the second elongate tubular body has a diameter of about 0.1 mm to about 400 mm.

6. The coaxial needle of any one of claims 3-5, further comprising a piercing member located on the distal end and attached to the first elongate tubular body.

7. The coaxial needle of any one of claims 3-5, further comprising a piercing member located on the distal end and attached to the second elongate tubular body.

8. The coaxial needle of claim 1 or 2, wherein the first elongate tubular body is arranged concentrically within the lumen of the second elongate tubular body.

9. The coaxial needle of claim 8, wherein the lumen of the first elongate tubular body has a diameter of 0.1 mm to 400 mm.

10. The coaxial needle of claim 8 or 9, wherein the lumen of the second elongate tubular body has a diameter of 0.5 mm to 500 mm.

11. The coaxial needle of any one of claims 8-10, further comprising a piercing member located on the distal end and attached to the first elongate tubular body.

12. The coaxial needle of any one of claims 8-10, further comprising a piercing member located on the distal end and attached to the second elongate tubular body.

13. The coaxial needle of any one of claims 1-12, wherein the plurality of side wall openings of the first elongate tubular body is arranged in a random, linear, spiral, radial, Fibonacci spiral, logarithmic spiral, Archimedean spiral, zig-zag, or checkerboard pattern.

14. The coaxial needle of any one of claims 1-12, wherein the plurality of openings of the first elongate tubular body are arranged in a pattern, wherein the pattern replicates the spacing of cells in a biological tissue.

15. The coaxial needle of any one of claims 1-14, wherein the plurality of side wall openings of the second elongate tubular body is arranged in a random, linear, spiral, radial, Fibonacci spiral, logarithmic spiral, Archimedean spiral, zig-zag, or checkerboard pattern.

16. The coaxial needle of any one of claims 1-14, wherein the plurality of openings of the second elongate tubular body are arranged in a pattern, wherein the pattern replicates the spacing of cells in a biological tissue.

17. The coaxial needle of any one of claims 1-16, wherein an area of the paired opening is at most 80 mm².

18. The coaxial needle of any one of claims 1-17, wherein the first elongate tubular body has a length of 0.5 cm to 200 cm.

19. The coaxial needle of any one of claims 1-18, wherein the second elongate tubular body has a length of 0.5 cm to 200 cm.

20. The coaxial needle of any one of claims 1-19, wherein at least one of the plurality of side wall openings of the first elongate tubular body has an elliptical shape, a circular shape, a rectangular shape, a triangular shape, or a tear drop shape.

21. The coaxial needle of any one of claims 1-20, wherein at least one of the plurality of side wall openings of the second elongate tubular body has an elliptical shape, a circular shape, a rectangular shape, a triangular shape, or a tear drop shape.

22. The coaxial needle of any one of claims 1-21, wherein at least one of the plurality of side wall openings of the first elongate tubular body or at least one of the plurality of side wall openings of the second elongate tubular body has an area of 0.1 mm² to 80 mm².

23. The coaxial needle of any one of claims 1-22, further comprising an opening located on the distal end.

24. The coaxial needle of any one of claims 1-23, further comprising an expanding member located on or adjacent to the distal end of the coaxial needle, wherein:
the expanding member is a metallic material; and
the metallic material comprises a shape memory alloy.

25. The coaxial needle of any one of claims 1-23, wherein the cylindrical side wall of the first elongate tubular body and the cylindrical side wall of the second elongate tubular body are a metallic material, wherein the metallic material comprises a shape memory alloy.

26. The coaxial needle of claim 24 or 25, wherein the shape memory alloy is nitinol.

27. The coaxial needle of any one of claims 1-26, wherein the second elongate tubular body is movable in the axial direction.

28. The coaxial needle of any one of claims 1-27, wherein the second elongate tubular body can rotate about the longitudinal axis of the second elongate tubular body.

29. A method of injecting fluid into a substrate comprising:
(a) inserting a coaxial needle into the substrate, wherein the coaxial needle comprises:
(I) a proximal end;
(II) a distal end; and
(III) an inner elongate tubular body and an outer elongate tubular body that are arranged concentrically, wherein the inner elongate tubular body and the outer elongate tubular body each comprise:
(i) a cylindrical side wall forming a lumen that extends along a longitudinal axis between the proximal end and the distal end; and
(ii) a plurality of side wall openings arranged along the cylindrical side wall;
wherein at least one of the inner or outer elongate tubular bodies is movable relative to the other to form a plurality of configurations, wherein at least one of the plurality of configurations comprises a paired opening formed by an overlap of at least one of the plurality of side wall openings of the inner elongate tubular body and at least one of the plurality of side wall openings of the outer elongate tubular body; and
(b) flowing a fluid into the lumen of the inner elongate tubular body such that the fluid passes through the paired opening and into the substrate.

30. The method of claim 29, wherein the paired opening is formed by one of the plurality of side wall openings of the first elongate tubular body and one of the plurality of sidewall openings of the second elongate tubular body.

31. The method of claim 29 or 30, wherein the inner elongate tubular body is movable relative to the outer elongate tubular body.

32. The method of claim 31, further comprising controlling a position of the inner elongate tubular body relative to the outer elongate tubular body.

33. The method of claim 31 or 32, wherein the inner elongate tubular body moves in the axial direction in relation to the outer elongate tubular body.

34. The method of any one of claims 31-33, wherein the inner elongate tubular body rotates about the longitudinal axis of the inner elongate tubular body.

35. The method of claim 29 or 30, wherein the outer elongate tubular body is movable relative to the inner elongate tubular body.

36. The method of claim 35, further comprising controlling a position of the outer elongate tubular body relative to the inner elongate tubular body.

37. The method of claim 35 or 36, wherein the outer elongate tubular body moves in the axial direction in relation to the inner elongate tubular body.

38. The method of any one of claims 35-37, wherein the outer elongate tubular body rotates about the longitudinal axis of the outer elongate tubular body.

39. The method of any one of claims 29-38, wherein the plurality of side wall openings of the inner elongate tubular body is arranged in random, linear, spiral, radial, Fibonacci spiral, logarithmic spiral, Archimedean spiral, zig-zag, or checkerboard pattern.

40. The method of any one of claims 29-38, wherein the plurality of openings of the inner elongate tubular body are arranged in a pattern, wherein the pattern replicates the spacing of cells in a biological tissue.

41. The method of any one of claims 29-40, wherein the plurality of side wall openings of the outer elongate tubular body is arranged in a random, linear, spiral, radial, Fibonacci spiral, logarithmic spiral, Archimedean spiral, zig-zag, or checkerboard pattern.

42. The method of any one of claims 29-40, wherein the plurality of openings of the outer elongate tubular body are arranged in a pattern, wherein the pattern replicates the spacing of cells in a biological tissue.

43. The method of any one of claims 29-42, wherein an area of the paired opening is at most 80 mm².

44. The method of any one of claims 29-43, wherein the fluid comprises a plurality of cells.

45. The method of claim 44, wherein the plurality of cells comprises chondrogenic cells, chondrogenic precursors, multipotent cells, or pluripotent cells.

46. The method of any one of claims 29-45, wherein the fluid flows through an opening located on the distal end of the coaxial needle.

47. The method of claim 46, wherein the opening located on the distal end of the coaxial needle increases in size after being inserted into the substrate.

48. The method of any one of claims 29-47, wherein the coaxial needle changes shape after being inserted into the substrate.

49. The method of any one of claims 29-48, wherein the substrate is a biological tissue.

50. The method of claim 49, wherein the biological tissue is human tissue, orthopedic tissue, cartilage, or devitalized tissue.

51. The method of any one of claims 29-48, wherein the substrate is an acellular scaffold.

52. A method of repairing a damaged tissue comprising:
(a) implanting an acellular tissue scaffold into the damaged tissue,
(b) inserting a coaxial needle into the acellular tissue scaffold, wherein the coaxial needle comprises:
(I) a proximal end;
(II) a distal end; and
(III) an inner elongate tubular body and an outer elongate tubular body that are arranged concentrically, wherein the inner elongate tubular body and the outer elongate tubular body each comprise:
(i) a cylindrical side wall forming a lumen that extends along a longitudinal axis between the proximal end and the distal end, and
(ii) a plurality of side wall openings arranged along the cylindrical side wall;
wherein at least one of the inner or outer elongate tubular bodies is movable relative to the other to form a plurality of configurations, wherein at least one of the plurality of configurations comprises a paired opening formed by an overlap of at least one of the plurality of side wall openings of the inner elongate tubular body and at least one of the plurality of side wall openings of the outer elongate tubular body; and
(c) flowing a fluid into the lumen of the inner elongate tubular body such that the fluid passes through the paired opening and into the acellular tissue scaffold.

53. The method of claim 52, wherein the paired opening is formed by one of the plurality of side wall openings of the first elongate tubular body and one of the plurality of sidewall openings of the second elongate tubular body.

54. The method of claim 52 or 53, wherein the inner elongate tubular body is movable relative to the outer elongate tubular body.

55. The method of claim 54, further comprising controlling a position of the inner elongate tubular body relative to the outer elongate tubular body.

56. The method of claim 54 or 55, wherein the inner elongate tubular body moves in the axial direction in relation to the outer elongate tubular body.

57. The method of any one of claims 54-56, wherein the inner elongate tubular body rotates about the longitudinal axis of the inner elongate tubular body.

58. The method of any one of claims 52-57, wherein the outer elongate tubular body is movable relative to the inner elongate tubular body.

59. The method of claim 58, further comprising controlling a position of the outer elongate tubular body relative to the inner elongate tubular body.

60. The method of claim 58 or 59, wherein the outer elongate tubular body moves in the axial direction in relation to the inner elongate tubular body.

61. The method of any one of claims 58-60, wherein the outer elongate tubular body rotates about the longitudinal axis of the outer elongate tubular body.

62. The method of any one of claims 52-61, wherein the plurality of side wall openings of the inner elongate tubular body is arranged in a random, linear, spiral, radial, Fibonacci spiral, logarithmic spiral, Archimedean spiral, zig-zag, or checkerboard pattern.

63. The method of any one of claims 52-61, wherein the plurality of openings of the inner elongate tubular body is arranged in a pattern, wherein the pattern replicates the spacing of cells in a biological tissue.

64. The method of any one of claims 52-63, wherein the plurality of side wall openings of the outer elongate tubular body is arranged in a random, linear, spiral, radial, Fibonacci spiral, logarithmic spiral, Archimedean spiral, zig-zag, or checkerboard pattern.

65. The method of any one of claims 52-63, wherein the plurality of openings of the outer elongate tubular body are arranged in a pattern, wherein the pattern replicates the spacing of cells in a biological tissue.

66. The method of any one of claims 52-65, wherein an area of the paired opening is at most 80 mm².

67. The method of any one of claims 52-66, wherein the fluid comprises a plurality of cells.

68. The method of claim 67, wherein the plurality of cells comprises chondrogenic cells, pluripotent cells, multipotent cells, or chondrogenic precursors.

69. The method of any one of claims 52-68, wherein the fluid flows through an opening located on the distal end of the coaxial needle.

70. The method of claim 69, wherein the opening located on the distal end of the coaxial needle increases in size after being inserted into the substrate.

71. The method of any one of claims 52-70, wherein the coaxial needle changes shape after being inserted into the substrate.

72. The method of any one of claims 52-71, wherein the acellular tissue scaffold is a devitalized tissue allograft.
